SEQUENCE LISTING (45 sequences)

SEQ ID NO: 1 - DNA, 52 bp, Artificial Sequence
  Human genome
gcatttctggctgagaggaagggagcrgctgggaaggtgccaccttgctggg

SEQ ID NO: 2 - DNA, 52 bp, Artificial Sequence
  Human genome
gcgcgtgcggaacatgaggctgaggtrccagggttgggagatgggcagcgag

SEQ ID NO: 3 - DNA, 52 bp, Artificial Sequence
  Human genome
cagggtgtgcgggtgagcgatacgccrgagggggttaacagcctggatccca

SEQ ID NO: 4 - DNA, 52 bp, Artificial Sequence
  human genome
taaggatccagggcaacgggcaggttntcaggtgcctggggccacatgctgg

SEQ ID NO: 5 - DNA, 52 bp, Artificial Sequence
  Human genome
ggccagagctcatgggttacacattgrcccagggtccccctgcgcctcagac

SEQ ID NO: 6 - DNA, 52 bp, Artificial Sequence
  Human genome
tcccagcagctagccctgctcctgcgmaacgccacgcagcacacagctggct

SEQ ID NO: 7 - DNA, 52 bp, Artificial Sequence
  Human genome
tgtgtgtgggtccaggcacagggctgrgaagctcttatgtagagaatgagga

SEQ ID NO: 8 - DNA, 52 bp, Artificial Sequence
  variation 27: n = c or t
tgtggccctcctttgctgtcctcctgntgctggggccgcgtgtcctcaggac

SEQ ID NO: 9 - DNA, 52 bp, Artificial Sequence
  variation 27: n = c or t
cagccatcccactccccacttactganctctctgttccctgcctagtcctac

SEQ ID NO: 10 - DNA, 52 bp, Artificial Sequence
  variation 27: n = c or t
tcagtgcggactcctccctaggaagangagaccatgctggaggggtcagcct

SEQ ID NO: 11 - DNA, 52 bp, Artificial Sequence
  variation 27: n = g or t
acagtgctggctcggctgccctgaggntgctcaatcaagcacaggtttcaag

SEQ ID NO: 12 - DNA, 52 bp, Artificial Sequence
  Human genome
gggactggacacgaacccatcccaacrcaaaaagcaaataaataaaacaaat

SEQ ID NO: 13 - DNA, 52 bp, Artificial Sequence
  Human genome
caacaaccaaactgtacaatctgatgrttagtaccaagttagcatccagcat

SEQ ID NO: 14 - DNA, 52 bp, Artificial Sequence
  Human genome
aaaaacaacaacaacaaaacgctaccmtatttacagcaacaaccaaactgta

SEQ ID NO: 15 - DNA, 52 bp, Artificial Sequence
  Human genome
ctatttgggagcagtgtcatggacatrggcagagggacaggcttatcagcca

SEQ ID NO: 16 - DNA, 52 bp, Artificial Sequence
  variation 27: n = g or t
ctacaccaaatctgttaaacgtgtctntgttattccttcaacaaacaccatc

SEQ ID NO: 17 - DNA, 52 bp, Artificial Sequence
  Human genome
cccagccccctgctccaatttctaacmaatttggagtaaatctctaattcca

SEQ ID NO: 18 - DNA, 52 bp, Artificial Sequence
  Human genome
aaagagaaagaaataggagcaggatcracttccagatatacagagaatataa

SEQ ID NO: 19 - DNA, 52 bp, Artificial Sequence
  variation 27: n = c or t
tggcattggttcactggacatttccangtgagcggcctccgtagctaacctc

SEQ ID NO: 20 - DNA, 52 bp, Artificial Sequence
  variation 27: n = c or t
gacaaaccacttgcctgtacttctcancttctatttgttcatttcactgctg

SEQ ID NO: 21 - DNA, 52 bp, Artificial Sequence
  variation 27: n = c or t
agaacggttgtgctgcctaacgtggcngctaaccgacgctctacgggaggaa

SEQ ID NO: 22 - DNA, 52 bp, Artificial Sequence
  Human genome
tatggacttgccctgaattatttcctrtgcaagggccaagaaccctctcttg

SEQ ID NO: 23 - DNA, 52 bp, Artificial Sequence
  Human genome
ccacctaaggccatctagtgccaactcccccccccaccattcccctcactgc

SEQ ID NO: 24 - DNA, 52 bp, Artificial Sequence
  Human genome
cacgcagccagggcttcacacccttcrggctgcacccgggcaggcctcagaa

SEQ ID NO: 25 - DNA, 52 bp, Artificial Sequence
  variation 27: n = c or t
ggtgaggggccagggcaaagggtgtgnctcgtcctgcccgcactgcctctcc

SEQ ID NO: 26 - DNA, 52 bp, Artificial Sequence; identical to SEQ ID NO: 11
  variation 27: n = g or t

SEQ ID NO: 27 - DNA, 52 bp, Artificial Sequence; identical to SEQ ID NO: 12
  Human genome

SEQ ID NO: 28 - DNA, 52 bp, Artificial Sequence
  Human genome
tgcaggaattgctgaggggagaagacagggggagaatccacggcagaaaagc

SEQ ID NO: 29 - DNA, 52 bp, Artificial Sequence; identical to SEQ ID NO: 14
  Human genome

SEQ ID NO: 30 - DNA, 52 bp, Artificial Sequence; identical to SEQ ID NO: 15
  Human genome

SEQ ID NO: 31 - DNA, 52 bp, Artificial Sequence
  variation 27: n = c or t
cctaggctcaagtgatcctcccgcctnggcctcccaaactggggtcacagat

SEQ ID NO: 32 - DNA, 52 bp, Artificial Sequence
  variation 27: n = g or t
agctgcgattacaggcatgtaccaccncacccagctaatttttttgtatttt

SEQ ID NO: 33 - DNA, 52 bp, Artificial Sequence
  Human genome
gatcgcaccactgcactccagcctggrtaatagagccagaccttgtctcaaa

SEQ ID NO: 34 - DNA, 52 bp, Artificial Sequence
  Human genome
agatgaaagttaagtgtagcttaattmacagtatgtgaggctattaagctag

SEQ ID NO: 35 - DNA, 52 bp, Artificial Sequence; identical to SEQ ID NO: 17
  Human genome

SEQ ID NO: 36 - DNA, 52 bp, Artificial Sequence
  variation 27: n = c, g or t
aaaaattagccggacgtgggtggcagncgcctgtaatctcagctactcggga

SEQ ID NO: 37 - DNA, 52 bp, Artificial Sequence
  Human genome
acagagcaagattctgtctaaaaaaaragaaagaaataggagcaggatcgac

SEQ ID NO: 38 - DNA, 52 bp, Artificial Sequence; identical to SEQ ID NO: 18
  Human genome

SEQ ID NO: 39 - DNA, 22 bp, Artificial Sequence
  oligonucleotides
ctaggcatattgcgcaatatgc

SEQ ID NO: 40 - DNA, 22 bp, Artificial Sequence
  oligonucleotides
gcatattgcgcaatatgcctag

SEQ ID NO: 41 - DNA, 21 bp, Artificial Sequence
  oligonucleotides
tgccctgaggttgctcaatca

SEQ ID NO: 42 - DNA, 21 bp, Artificial Sequence
  oligonucleotide
tgattgagcaacctcagggca

SEQ ID NO: 43 - DNA, 21 bp, Artificial Sequence
  oligonucleotide
tgccctgagggtgctcaatca

SEQ ID NO: 44 - DNA, 21 bp, Artificial Sequence
  oligonucleotide
tgattgagcaccctcagggca

SEQ ID NO: 45 - DNA, 35 bp, Artificial Sequence
  Mouse genome
tggcatggtggccctgagggggccccagcacaggt

(19) United States
(12) Patent Application Publication
(10) Pub. No.: US 2012/0289593 A1
(43) Pub. Date: Nov. 15, 2012

(54) METHODS FOR RISK ASSESSMENT, TREATING, AND DIAGNOSING MYOCARDIAL INFARCTION

(75) Inventors: Sekar Kathiresan, Newton, MA (US); Kiran Musunuru, Cambridge, MA (US)

(73) Assignee: THE GENERAL HOSPITAL CORPORATION, Boston, MA (US)

(21) Appl. No.: 13/394,716

(22) PCT Filed: Sep. 8, 2010
(86) PCT No.: PCT/US2010/048142

§ 371 Date: Jun. 28, 2012

Related U.S. Application Data
(60) Provisional application No. 61/240,470, filed on Sep. 8, 2009.
(60) Provisional application No. 61/365,489, filed on Jul. 19, 2010.

(51) Int. Cl. C12Q 1/68; A61P 7/02; A61P 9/08; A61K 31/21; A61P 3/06

(52) U.S. Cl. 514/509; 435/6.11

(57) ABSTRACT

This document features method related to genetic markers associated with myocardial infarction. For example, methods of using such genetic markers for risk assessment and for diagnosing and treating myocardial infarction are provided.

### CLAIM OF PRIORITY

This application claims the benefit of U.S. Provisional Patent Applications Ser. Nos. 61/240,470, filed on Sep. 8, 2009, and 61/365,489, filed on Jul. 19, 2010. The entire contents of the foregoing are hereby incorporated by reference.

### TECHNICAL FIELD

This document features methods related to genetic markers of susceptibility to cardiovascular disease. For example, this document provides methods of using such genetic markers for risk assessment and for diagnosing and treating cardiovascular diseases such as myocardial infarction.

### BACKGROUND

Cardiovascular diseases are diseases that involve the heart or blood vessels. Among the cardiovascular diseases are atherosclerosis, congestive heart failure, myocardial infarction, and peripheral vascular disease. Myocardial infarction (MI) is the leading cause of death in the developed world. There is an established link between MI and cholesterol levels. For example, individuals with the Mendelian disorder of familial hypercholesterolemia are more susceptible to MI. Moreover, medications that lower low-density lipoprotein cholesterol have been successfully used to reduce the incidence of MI in clinical trials in a variety of populations. However, traditional screening methods for determining risk of MI and cardiovascular disease such as the Framingham Risk Profile and standard lipid panels, which measure triglycerides, high-density lipoprotein (HDL), low-density lipoprotein (LDL), and total cholesterol, may not accurately assess MI and cardiovascular disease risk.

Cholesterol, a building block of cell membranes, is transported through the blood in the form of water-soluble carrier molecules known as lipoproteins. LDL contributes 60% to 70% of the total serum cholesterol. There is considerable heterogeneity among cholesterol-carrying LDL particles, ranging from very small, dense, lipid-depleted particles to large, buoyant, cholesterol-enriched particles. LDL cholesterol (LDL-C) can be separated into seven different kinds of particles, and based on the size of these particles, two LDL subclasses have been created: LDL subclass A and LDL subclass B. The particles in LDL subclass B are smaller and more dense than those found in LDL subclass A. Increased cardiovascular risk has been associated with these LDL-C subclasses. For example, studies have suggested that small LDL particles may carry disproportionate atherosclerotic risk. The mechanisms that generate very small and small LDL particles remain poorly understood. There also is a need for additional methods of reducing LDL-C. Despite aggressive use of LDL-C-lowering medications such as statins, many individuals do not achieve the LDL-C levels recommended by clinical guidelines. In addition, no pharmacological therapies specifically target the LDL subclasses. There remains a need to more conclusively identify individuals at risk for developing cardiovascular diseases and to select and optimize appropriate therapies based on an individual's genotypic subtype.

### SUMMARY

This document describes genetic variations involved in lipoprotein metabolism and associated with myocardial infarction (MI) and other cardiovascular diseases (e.g., atherosclerosis, hypercholesterolemia). For example, methods of selecting and administering optimal treatments for MI or other cardiovascular diseases, based on the presence or absence of allelic and genotypic variants identified herein are also provided. As described herein, this document provides methods and materials by which clinicians and other professionals can detect single nucleotide polymorphisms (SNP) at the chromosome 1p13 locus described herein that are associated with myocardial infarction, atherosclerosis, and/or hypercholesterolemia and use such information for risk assessment, diagnosis, and selecting and optimizing treatment options. Such diagnostic and therapeutic methods can have substantial value for clinical use.

In one aspect, this document features a method of predicting a human patient's likelihood of developing a cardiovascular disorder. The method can comprise determining the identity of at least one allele of a single nucleotide polymorphism at rs12740374, wherein the presence of an allele associated with a cardiovascular disorder (i.e., an allele associated with an increased risk of developing the disorder) indicates that the subject has an increased risk of developing the cardiovascular disorder, and wherein the presence of an alternative allele indicates that the subject has a decreased (or normal or not increased) risk of developing the cardiovascular disorder. The cardiovascular disorder can be myocardial infarction or elevated levels of low-density lipoprotein cholesterol (LDL-C). The allele associated with a cardiovascular disorder can be a “G” at nucleotide 27 of SEQ ID NO:11. Determining the identity of an allele can comprise obtaining a sample comprising DNA from the patient, and determining identity of the nucleotide at rs12740374. Determining the identity of the nucleotide can comprise contacting the sample with a probe, e.g., a probe specific for a selected allele of the polymorphism, and detecting the formation of complexes between the probe and the selected allele of the polymorphism, wherein the formation of complexes between the probe and the test marker indicates the presence of the selected allele in the sample. The method can further comprise selecting a treatment method based on the identity of an allele at rs12740374. The treatment can comprise administration of a medicament selected from the group consisting of a hypolipidemic medication, a vasodilating compound, an anticoagulant, and sublingual glyceryl trinitrate, or any combination thereof. The method can further comprise administering the selected treatment to the subject. The method can further comprise recording the identity of the allele in a tangible medium. The tangible medium can comprise a computer readable disk, a solid state memory device, or an optical storage device.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-D are graphs demonstrating preferential association of human chromosome 1p13 locus with very small LDL and liver gene expression. (1A) Mean plasma lipid and lipoprotein levels in homozygotes for the minor haplotype of the 1p13 locus (minor allele of rs646776) vs. homozygotes for the major haplotype (major allele of rs646776), normalized to the mean level in minor haplotype homozygotes, in the Malmö Diet and Cancer Study—Cardiovascular Cohort (MDC-CC) (measured by ion mobility) and the Pharmacogenomics and Risk of Cardiovascular Disease (PARC) cohort (measured by gradient gel electrophoresis). LDL-L=large LDL; LDL-M=medium LDL; LDL-S=small LDL; LDL-VS=very small LDL. (1B) Relative gene positions in and around the 1p13 locus; * indicates position of rs646776. (1C) Mean expression of local genes in homozygotes for the minor 1p13 haplotype (minor allele of rs646776) vs. heterozygotes vs. homozygotes for the major 1p13 haplotype (major allele of rs646776), normalized to the mean level in minor haplotype homozygotes, in samples of human liver, human subcutaneous adipose, and human omental adipose. (1D) Mean expression of PSRC1, CELSR2, SORT1, and TCF7L2 (negative control) mRNA, standardized to B2M expression, and sortilin protein, standardized to a-tubulin, in samples of human liver from homozygotes for the minor 1p13 haplotype (minor allele of rs12740374) vs. heterozygotes vs. homozygotes for the major 1p13 haplotype (major allele of rs12740374), normalized to the mean level in minor haplotype homozygotes. P values derived from linear regression analyses or unpaired t test. Error bars show s.e.m.

FIG. 2 shows sortilin and α-tubulin expression by immunoblot in human liver lysates of various genotypes at rs12740374.

FIGS. 3A-C are graphs demonstrating rs12740374 haplotype-specific difference in transcriptional activity. (3A) Map of 1p13 SNPs genotyped in ˜20,000 individuals of European descent relative to CELSR2 and PSRC1 genes. The six SNPs with strongest association with LDL-C (indicated with boxes), comprising a single haplotype, define the 6.1 kb region between the stop codons of the two genes. (3B) Firefly luciferase expression from constructs transfected into Hep3B human hepatoma cells. Both the major (darker colors) and minor (lighter colors) haplotypes of the 6.1 kb region were subcloned in forward and reverse orientations into a basa1 firefly luciferase construct with the SV40 promoter. Shown are ratios of firefly luciferase expression to Renilla luciferase expression (expressed from cotransfected plasmid), measured 48 hours after transfection, normalized to the mean ratio from the major haplotype, forward orientation construct. Error bars show s.e.m., N=2. (3C) Both the major and minor haplotypes of a minimal 2.1 kb region were subcloned into the basal construct. Single nucleotide alterations were introduced into the minor haplotype, changing minor alleles of SNPs into major alleles. Shown are ratios of firefly luciferase expression to Renilla luciferase expression normalized to the mean ratio from the major haplotype construct. Error bars show s.e.m., N=4.

FIG. 4 is a diagram depicting linkage disequilibrium patterns for SNPs in the vicinity of rs12740374 in Europeans (HapMap CEU, release 21) and Africans (HapMap CEU, release 21). Diagrams were generated with Haploview version 4.1. In each square, the intensity of red shading is proportional to D′ between SNPs; the number is r² between SNPs (if no number is listed, r²=1.0). The six SNPs with strongest association with LDL-C in Europeans are indicated with boxes. Haplotypes for the six SNPs in Europeans and Africans were also generated with Haploview.

FIGS. 5A-F are schematics showing alteration of a C/EBP transcription factor consensus binding site by rs12740374. (5A) The human DNA sequence surrounding rs12740374, major and minor alleles, and orthologous DNA sequence in mouse. The major allele of rs12740374 disrupts one of two core elements (position 2-3, 8-9) in the predicted consensus binding site on which a C/EBP dimer binds. (5B) Electrophoretic mobility shift assays (EMSA) with labeled probes matching the C/EBP consensus binding site, the rs12740374 minor allele (T) sequence, and the rs12740374 major allele (G) sequence. Competition assays were performed with 100-fold excess of cold probe. Either of two C/EBPα antibodies was used to compete for binding and/or shift the protein-DNA complex. (5C) Relative firefly luciferase expression from constructs with haplotypes of 2.1 kb region transfected into Hep3B cells. Single nucleotide alterations were introduced into constructs as indicated, altering rs12740374 and the three other core recognition nucleotides in the predicted C/EBP consensus binding site. (5D and 5E) Relative firefly luciferase expression from constructs with haplotypes of 6.1 kb region transfected into (D) Hep3B human hepatoma cells with or without concomitant transduction with A-C/EBP (dominant negative C/EBP) cDNA via lentivrus and (E) NIH 3T3 fibroblasts with or without concomitant transduction with C/EBP-α cDNA via lentivirus. (5F) Relative SORT1 expression, determined as a ratio with B2M expression by qRT-PCR, in Hep3B cells (homozygous major (GG) at rs12740374) or SK-HEP-1 human hepatoma cells (heterozygous (GT) at rs12740374) with or without concomitant transduction with A-C/EBP cDNA via lentivirus. Error bars show s.e.m., N=3 for each experiment.

FIGS. 6A-D depict a map of 1p13 SNPs genotyped in 20,000 individuals of European descent relative to CELSR2 and PSRC1 genes. (6A) The six SNPs with strongest association with LDL-C (indicated with boxes), comprising a single haplotype, define the 6.1 kb region between the stop codons of the two genes. (6B) Firefly luciferase expression from constructs with haplotypes of 2.1 kb region transfected into Hep3B human hepatoma cells with or without concomitant transduction with A-C/EBP (dominant negative C/EBP) cDNA via lentivrus. Shown are ratios of firefly luciferase expression to Renilla luciferase expression (expressed from cotransfected plasmid), measured 48 hours after transfection, normalized to the ratio from the 2.1 kb major haplotype construct in the absence of A-C/EBP. (6C) Chromatin immunoprecipitation with antibody against C/EBPα in HUES-1 human embryonic stem cells (homozygous minor (TT) at rs12740374)with transduction with C/EBPα cDNA via lentivirus Immunoprecipitation of DNA sequence surrounding rs12740374 was measured by quantitative PCR, relative to 1:30 dilution of input chromatin, normalized to background (control condition with IgG beads alone, with no antibody). (6D) Relative SORT1 expression, determined as a ratio with B2M expression by qRT-PCR, in HUES-1 or HUES-9 cells (heterozygous (GT) at rs12740374) either maintained in a pluripotent state or differentiated into endodermal cells with EndoMedia (RPMI-B27 media supplemented with activin A), with or without concomitant transduction C/EBPα cDNA via lentivirus. Error bars show s.e.m., N=3 for each experiment.

FIGS. 7A-M contain graphs demonstrating altered plasma lipids and lipoproteins following overexpression or knockdown of Sort1 in mouse liver. Adeno-associated virus 8 (AAV8) vectors either containing no gene, murine Sort1 cDNA, truncated Sort1 cDNA, or murine Psrc1 cDNA were administered via intraperitoneal injection; phosphate-buffered saline or siRNA duplex targeting mouse Sort1 and prepared in lipidoid formulation were administered weekly at 2.0 mg/kg via tail vein injection. Plasma samples were collected prior to injection and at various timepoints after injection, and were subjected: individually to analytical chemistry (Mira autoanalyzer) to measure total cholesterol (7A, E, G, I); as pooled samples to FPLC (7D, F, H, J), from which fractions 10 to 26 were used to calculate LDL-C levels (7A, E, G, I); individually to NMR to measure LDL particle concentrations (7B). P values calculated with unpaired t test, shown if P<0.05. Error bars show s.e.m. (7A-D) Apobec1−/−; APOB Tg mice (five mice per group). (7B) NMR measurements at six weeks. LDL-L=large LDL; LDL-M/S=medium small LDL; LDL-VS=very small LDL. (7C) The mice were intraperitoneally injected with Pluronic F-127 detergent to block VLDL triglyceride lipolysis and permit assessment of the rate of VLDL secretion. Plasma samples were collected at baseline, one hour, two hours, and four hours after injection. VLDL particle concentrations were measured from pooled samples with NMR. (7E, F) Apobec1−/−; APOB Tg mice (five mice per group). (7G, H) Ldlr−/− mice (five mice per group). (7I, J) Apobec1−/−; APOB Tg; Ldlr−/−mice (five mice per group). (7K, L), Sortilin and actin expression by Western blot in liver and adipose samples from mice receiving (7K) AAV8 vectors either containing no gene or murine Sort1 cDNA or (7L) control or Sort1 siRNA injections. (7M) Table summarizing the results of all Sort1 overexpression or knockdown experiments. Each Δ measurement indicates the difference between the experimental mice (mean) and control mice (mean) at the listed timepoint after injection.

FIGS. 8A-H are graphs and images demonstrating altered secretion of apoB with sortilin overexpression in hepatocytes. Primary mouse hepatocytes from Apobec1−/−; APOB Tg; Ldlr+/− mice (8A-D, F) or Apobec1−/−; Ldlr−/− mice (8E) were labeled for three hours, followed by collection of media, immunoprecipitation of apoB, polyacrylamide gel electrophoresis, and quantitation of radioactive counts from bands corresponding to apoB-100, as well as counts from trichloroacetic acid precipitation of media to determine total secreted protein levels. ApoB-100 measurements were standardized to total secreted protein measurements. (8A) Labeled apoB-100 secretion from hepatocytes treated with siRNA duplexes targeting luciferase or mouse Sort1. (8B) Sortilin and actin expression by Western blot for experiment shown in A. (8C) Autoradiograph for experiment shown in A. (8D, E) Labeled apoB-100 secretion from hepatocytes infected with adeno-associated virus 8 (AAV8) vectors either containing no gene or murine Sort1 cDNA, incubated with or without E64d (10 μM), an endolysosomal cathepsin inhibitor. (8F) Autoradiograph for experiment shown in D. (8G) Surface plasmon resonance sensorgrams of the interaction of human LDL with immobilized human sortilin, demonstrating the rapid association and slow dissociation phases. Quantitative analysis of the sensorgrams using a two-state binding model yields a K_{d}˜2 nM for the sortilin-apoB interaction. (8H) Immunoprecipitation of sortilin with apoB antibody and apoB with sortilin antibody in HuH-7 cells transfected with SORT1.

FIGS. 9A-C are a series of graphs showing the effects of overexpression of Sort1 in mouse livers. Adeno-associated virus 8 (AAV8) vectors either containing no gene or murine Sort1 cDNA were administered to five Apobec1−/−; APOB Tg mice each via intraperitoneal injection. Plasma samples were collected at two and six weeks after injection. The mice were sacrificed and liver tissue collected at six weeks. FIGS. 9A and 9B are graphs showing results from plasma samples that were subjected: individually to analytical chemistry (Mira) to measure ALT and total cholesterol at two and six weeks; as pooled samples to FPLC to measure LDL-C at two weeks; and individually to NMR to measure LDL particle concentrations at six weeks. LDL-L=large LDL; LDL-M/S=medium small LDL; LDL-VS=very small LDL. FIG. 9C is a graph showing results from plasma samples taken from mice that had received AAV vectors and were intraperitoneally injected with Pluronic F-127 detergent to block VLDL triglyceride lipolysis and permit assessment of the rate of VLDL secretion. Plasma samples were collected at baseline, one hour, two hours, and four hours after injection. VLDL particle concentrations were measured with NMR. P values calculated with unpaired t test, shown if P<0.05. Error bars show s.e.m.

FIG. 10 is a schematic depicting a proposed model for the 1p13-sortilin pathway. Hepatic apoB synthesis and lipidation begins in the endoplasmic reticulum (ER). When insufficient lipid is available to lipidate nascent apoB, apoB undergoes proteasome-mediated degradation (ERAD). Bulk lipid addition continues in the Golgi apparatus to form triglyceride-rich VLDL. Golgi localized apoB may either be transported for secretion across the plasma membrane or targeted for lysosomal degradation (PERPP); sortilin may be the primary trafficking receptor that “sorts” apoB/VLDL to the lysosome. VLDL that is secreted undergoes extrahepatic lipolysis into LDL. In hepatocytes with the minor allele (T) at rs12740374, this DNA base creates a consensus site on which a dimer of a C/EBP-related protein binds, which in turns activates transcription of the SORT1 gene in the nucleus. This results in increased production of sortilin protein, which may shunt lapidated apoB particles along the PERPP pathway, thereby resulting in decreased apoB/VLDL secretion.

### DETAILED DESCRIPTION

The present invention is based at least in part on the discovery that a single noncoding DNA variant at the chromosome 1p13 locus influences LDL-C and MI risk via liver-specific transcriptional regulation of the SORT1 gene. For example, the minor haplotype at the 1p13 locus is associated with substantially decreased plasma LDL-C and LDL-VS concentrations in humans and is correlated with a four- to thirteen-fold increase in SORT1 expression specifically in human liver. Increased Sort1 expression in mouse hepatocytes decreases apoB secretion, while increased SORT1 expression is associated with decreased plasma LDL-C and LDL-VS concentrations. These results suggest a novel biological pathway of lipoprotein regulation. Based at least in part on these discoveries are methods for assessing genetic risk based on evaluation of single nucleotide polymorphisms (SNPs) for genes at the chromosome 1p13 locus relating to cardiovascular diseases such as myocardial infarction, atherosclerosis, and/or hypercholesterolemia.

### Definitions

As used herein, an “allele” is one of a pair or series of genetic variants of a polymorphism at a specific genomic location. A “response allele” is an allele that is associated with altered response to a treatment. Where a SNP is biallelic, both alleles will be response alleles (e.g., one will be associated with a positive response, while the other allele is associated with no or a negative response, or some variation thereof).

As used herein, “genotype” refers to the diploid combination of alleles for a given genetic polymorphism. A homozygous subject carries two copies of the same allele and a heterozygous subject carries two different alleles.

As used herein, a “haplotype” is one or a set of signature genetic changes (polymorphisms) that are normally grouped closely together on the DNA strand, and are usually inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minisatellites.

Microsatellites (sometimes referred to as a variable number of tandem repeats or VNTRs) are short segments of DNA that have a repeated sequence, usually about 2 to 5 nucleotides long (e.g., CACACA), that tend to occur in non-coding DNA. Changes in the microsatellites sometimes occur during the genetic recombination of sexual reproduction, increasing or decreasing the number of repeats found at an allele, changing the length of the allele. Microsatellite markers are stable, polymorphic, easily analyzed and occur regularly throughout the genome, making them especially suitable for genetic analysis.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

The term “label” or “label containing moiety” refers in a moiety capable of detection, such as a radioactive isotope or group containing same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “direct label probe” (or “directly labeled probe”) refers to a nucleic acid probe whose label after hybrid formation with a target is detectable without further reactive processing of hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The terms “target,” “DNA target,” or “DNA target region” refers to a nucleotide sequence that occurs at a specific chromosomal location. Each such sequence or portion is preferably at least partially, single stranded (e.g., denatured) at the time of hybridization. When the target nucleotide sequences are located only in a single region or fraction of a given chromosome, the term “target region” is sometimes used. Targets for hybridization can be derived from specimens which include, but are not limited to, chromosomes or regions of chromosomes in normal, diseased or malignant human cells, either interphase or at any state of meiosis or mitosis, and either extracted or derived from living or postmortem tissues, organs or fluids; germinal cells including sperm and egg cells, or cells from zygotes, fetuses, or embryos, or chorionic or amniotic cells, or cells from any other germinating body; cells grown in vitro, from either long-term or short-term culture, and either normal, immortalized or transformed; inter- or intraspecific hybrids of different types of cells or differentiation states of these cells; individual chromosomes or portions of chromosomes, or translocated, deleted or other damaged chromosomes, isolated by any of a number of means known to those with skill in the art, including libraries of such chromosomes cloned and propagated in prokaryotic or other cloning vectors, or amplified in vitro by means well known to those with skill; or any forensic material, including but not limited to blood, or other samples.

The term “hybrid” refers to the product of a hybridization procedure between a probe and a target.

The term “hybridizing conditions” has general reference to the combinations of conditions that are employable in a given hybridization procedure to produce hybrids, such conditions typically involving controlled temperature, liquid phase, and contact between a probe (or probe composition) and a target. Conveniently and preferably, at least one denaturation step precedes a step wherein a probe or probe composition is contacted with a target. Guidance for performing hybridization reactions can be found in Ausubel et al., Current Protocols in Molecular Biology, John Wiley & Sons, N.Y. (2003), 6.3.1-6.3.6. Aqueous and nonaqueous methods are described in that reference and either can be used. Hybridization conditions referred to herein are a 50% formamide, 2×SSC wash for 10 minutes at 45° C. followed by a 2×SSC wash for 10 minutes at 37° C.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 30% (e.g., at least 40%, 50%, 60%, 70%, 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the GAP program in the GCG software package, using a Blossum 62 scoring matrix with a gap penalty of 12, a gap extend penalty of 4, and a frameshift gap penalty of 5.

As used herein, the term “substantially identical” is used to refer to a first nucleotide sequence that contains a sufficient number of identical nucleotides to a second nucleotide sequence such that the first and second nucleotide sequences have similar activities. Nucleotide sequences that are substantially identical are at least 80% (e.g., 85%, 90%, 95%, 97% or more) identical.

The term “nonspecific binding DNA” refers to DNA that is complementary to DNA segments of a probe, which DNA occurs in at least one other position in a genome, outside of a selected chromosomal target region within that genome. An example of nonspecific binding DNA comprises a class of DNA repeated segments whose members commonly occur in more than one chromosome or chromosome region. Such common repetitive segments tend to hybridize to a greater extent than other DNA segments that are present in probe composition.

As used herein, “determining the identity of an allele” includes obtaining information regarding the identity (i.e., of a specific nucleotide), presence or absence of one or more specific alleles in a subject. Determining the identity of an allele can, but need not, include obtaining a sample comprising DNA from a subject, and/or assessing the identity, presence or absence of one or more genetic markers in the sample. The individual or organization who determines the identity of the allele need not actually carry out the physical analysis of a sample from a subject; the methods can include using information obtained by analysis of the sample by a third party. Thus the methods can include steps that occur at more than one site. For example, a sample can be obtained from a subject at a first site, such as at a health care provider, or at the subject's home in the case of a self-testing kit. The sample can be analyzed at the same or a second site, e.g., at a laboratory or other testing facility.

As used herein, the term “response” refers to a qualitative or quantitative change in a subject with respect to onset, progression, symptomatology, recurrence, or severity of an SSD following a method of treating such a disorder. The term “response haplotype” means a haplotype that is associated with a particular response to a given drug.

### Detecting Polymorphism Variants

This document provides methods for assessing genetic risk of myocardial infarction, atherosclerosis, and/or hypercholesterolemia (e.g., elevated LDL-C) based on evaluation of single nucleotide polymorphisms (SNPs) or haplotypes at a chromosome 1p13 locus as described herein. Genes at the chromosome 1p13 locus include SORT1 (sortilin); CELSR2 (cadherin EGF LAG seven-pass G-type receptor 2); PSRC1 (proline/serine-rich coiled-coil 1); APOB (apolipoprotein B); PSMA5 (proteasome subunit, alpha type 5); SARS (seryl-tRNA synthetase); MYBPHL (myosin binding protein H-like); and SYPL2 (synaptophysin-like 2). One or more of the following exemplary SNPs can be used to capture significant haplotype variation in these genes: rs12740374; rs646776; rs599839; rs660240; rs629301; and/or rs602633. Other SNPs that can be used to capture haplotype variation include rs7528419; rs660240; rs4970833; rs653635; rs6689614; rs6657811; rs608196; rs2281894; rs17035630; rs17035665; rs4970834; rs611917; rs658435; rs17035949; rs10410; rs14000; rs657420; rs672569; rs11102967;. rs3832016; rs3902354; rs583104; rs4970837; and/or rs1277930.

Important variants can be identified via TDT using families with multiple affected individuals (such as those collected CCGS) and verified by Case/Control comparisons using the SNP markers presented herein. Using SNP markers identical to or in linkage disequilibrium with the exemplary SNPs, one can determine the haplotypes in these genes relating to genetic risk of developing myocardial infarction, atherosclerosis, and/or hypercholesterolemia via family-based and population-based association analyses. These haplotypes can then be used to determine risk of developing myocardial infarction by Case/Control studies. The allelic and genotypic variants thus identified can be used for assessing genetic risk.

As used herein, “obtaining a haplotype” includes obtaining information regarding the identity, presence or absence of one or more genetic markers in a subject. Obtaining a haplotype can, but need not, include obtaining a sample comprising DNA from a subject, and/or assessing the identity, presence or absence of one or more genetic markers in the sample. The individual or organization who obtains the haplotype need not actually carry out the physical analysis of a sample from a subject; the haplotype can include information obtained by analysis of the sample by a third party. Thus the methods can include steps that occur at more than one site. Obtaining a haplotype can also include or consist of reviewing a subject's medical history, where the medical history includes information regarding the identity, presence or absence of one or more genetic markers in the subject, e.g., results of a genetic test.

In some embodiments, to obtain a haplotype described herein, a biological sample that includes nucleated cells (such as blood, a cheek swab or mouthwash) is prepared and analyzed for the presence or absence of preselected markers. Such haplotype determinations may be performed by diagnostic laboratories, or, alternatively, diagnostic kits can be manufactured and sold to health care providers or to private individuals for self-diagnosis. Diagnostic or prognostic tests can be performed as described herein or using well known techniques, such as described in U.S. Pat. No. 5,800,998. The presence or absence of the haplotype in a patient may be ascertained by using any of the methods described herein. In some cases, results of these tests, and optionally interpretive information, can be returned to the subject or the health care provider. Information gleaned from the methods described herein can also be used to select or stratify subjects for a clinical trial. For example, the presence of a selected haplotype described herein can be used to select a subject for a trial.

### Linkage Disequilibrium Analysis

Linkage disequilibrium (LD) is a measure of the degree of association between alleles in a population. One of skill in the art will appreciate that haplotypes involving markers in LD with the polymorphisms described herein can also be used in a similar manner to those described herein. Methods of calculating LD are known in the art (see, e.g., Morton et al., Proc. Natl. Acad. Sci. USA 98(9): 5217-21 (2001); Tapper et al., Proc. Natl. Acad. Sci. USA 102(33): 11835-11839 (2005); Maniatis et al., Proc. Natl. Acad. Sci. USA 99: 2228-2233 (2002)). Thus, in some cases, the methods can include analysis of polymorphisms that are in LD with a polymorphism described herein. Methods are known in the art for identifying such polymorphisms; for example, the International HapMap Project provides a public database that can be used (see HapMap.org on the World Wide Web) as well as The International HapMap Consortium, Nature 426: 789-796 (2003), and The International HapMap Consortium, Nature 437: 1299-1320 (2005). Generally, it will be desirable to use a HapMap constructed using data from individuals who share ethnicity with the subject. For example, a HapMap for African-Americans would ideally be used to identify markers in LD with an exemplary marker described herein for use in genotyping a subject of African-American descent.

Alternatively, methods described herein can include analysis of polymorphisms that show a correlation coefficient (r²) of value≧0.5, e.g., an r²=1, with the markers described herein. Results can be obtained from on-line public resources such as HapMap.org on the World Wide Web. The correlation coefficient is a measure of LD, and reflects the degree to which alleles at two loci (for example, two SNPs) occur together, such that an allele at one SNP position can predict the correlated allele at a second SNP position, in the case where r² is >0.

### Identifying Additional Genetic Markers

In general, genetic markers can be identified using any of a number of methods well known in the art. For example, numerous polymorphisms in the regions described herein are known to exist and are available in public databases, which can be searched using methods and algorithms known in the art. Alternately, polymorphisms can be identified by sequencing either genomic DNA or cDNA in the region in which it is desired to find a polymorphism. According to one approach, primers are designed to amplify such a region, and DNA from a subject is obtained and amplified. The DNA is sequenced, and the sequence (referred to as a “subject sequence” or “test sequence”) is compared with a reference sequence, which can represent the “normal” or “wild type” sequence, or the “affected” sequence. In some embodiments, a reference sequence can be from, for example, the human draft genome sequence, publicly available in various databases, or a sequence deposited in a database such as GenBank. In some embodiments, the reference sequence is a composite of ethnically diverse individuals.

In general, if sequencing reveals a difference between the sequenced region and the reference sequence, a polymorphism has been identified. The fact that a difference in nucleotide sequence is identified at a particular site that determines that a polymorphism exists at that site. In most instances, particularly in the case of SNPs, only two polymorphic variants will exist at any location. However, in the case of SNPs, up to four variants may exist since there are four naturally occurring nucleotides in DNA. Other polymorphisms, such as insertions and deletions, may have more than four alleles.

The methods described herein can also include determining the presence or absence of other markers known or suspected to be associated with myocardial infarction, e.g., markers outside of a region identified herein, including, for example, markers on chromosome 1 and other chromosomes, e.g., in the region of 1p13. In some cases, the methods include determining the presence or absence of one or more other markers that are or may be associated with myocardial infarction, atherosclerosis, and/or hypercholesterolemia, e.g., in one or more genes.

The methods described herein include determining the presence or absence of haplotypes associated with myocardial infarction, atherosclerosis, and/or hypercholesterolemia. In some embodiments, an association with myocardial infarction, atherosclerosis, and/or hypercholesterolemia is determined by the presence of a shared haplotype between the subject and an affected reference individual, e.g., a first or second-degree relation of the subject, and the absence of the haplotype in an unaffected reference individual. Thus the methods can include obtaining and analyzing a sample from a suitable reference individual. Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine or expectorant. The sample itself will typically consist of nucleated cells (e.g., blood or buccal cells) or tissue removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al., Current Protocols in Molecular Biology, eds., John Wiley & Sons (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The absence or presence of a haplotype associated with myocardial infarction, atherosclerosis, and/or hypercholesterolemia as described herein can be determined using methods known in the art. For example, gel electrophoresis, capillary electrophoresis, size exclusion chromatography, sequencing, and/or arrays can be used to detect the presence or absence of the marker(s) of the haplotype. Amplification of nucleic acids, where desirable, can be accomplished using methods known in the art, e.g., PCR. In one example, a sample (e.g., a sample comprising genomic DNA), is obtained from a subject. The DNA in the sample is then examined to determine a haplotype as described herein. The haplotype can be determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

Other methods of nucleic acid analysis can include direct manual sequencing (Church and Gilbert, Proc. Natl. Acad. Sci. USA 81: 1991-1995 (1988); Sanger et al., Proc. Natl. Acad. Sci. USA 74: 5463-5467 (1977); Beavis et al., U.S. Pat. No. 5,288,644); automated fluorescent sequencing; single-stranded conformation polymorphism assays (SSCP) (Schafer et al., Nat. Biotechnol. 15: 33-39 (1995)); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86: 232-236 (1989)); denaturing high performance liquid chromatography (DHPLC, Underhill et al., Genome Res. 7: 996-1005 (1997)); infrared matrix-assisted laser desorption/ionization (IR-MALDI) mass spectrometry (WO 99/57318); mobility shift analysis (Orita et al., Proc. Natl. Acad. Sci. USA 86: 2766-2770 (1989)); restriction enzyme analysis (Flavell et al., Cell 15: 25 (1978); Geever et al., Proc. Natl. Acad. Sci. USA 78: 5081 (1981)); quantitative real-time PCR (Raca et al., Genet Test 8(4): 387-94 (2004)); heteroduplex analysis; chemical mismatch cleavage (CMC) (Cotton et al., Proc. Natl. Acad. Sci. USA 85: 4397-4401 (1985)); RNase protection assays (Myers et al., Science 230: 1242 (1985)); use of polypeptides that recognize nucleotide mismatches, e.g., E. coli mutS protein; allele-specific PCR, and combinations of such methods. See, e.g., Gerber et al., U.S. Pat. Publication No. 2004/0014095 which is incorporated herein by reference in its entirety.

Sequence analysis can also be used to detect specific polymorphic variants. For example, polymorphic variants can be detected by sequencing exons, introns, 5′ untranslated sequences, or 3′ untranslated sequences. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined. Real-time pyrophosphate DNA sequencing is yet another approach to detection of polymorphisms and polymorphic variants (Alderborn et al., Genome Research 10(8): 1249-1258 (2000)). Additional methods include, for example, PCR amplification in combination with denaturing high performance liquid chromatography (dHPLC) (Underhill et al., Genome Research 7(10): 996-1005 (1997)).

In some embodiments, the methods described herein include determining the sequence of the entire region of the CELSR2 locus described herein as being of interest, e.g., between and including SNPs rs4970833 and rs629301. In some embodiments, the methods described herein include determining the sequence of the entire region of the PSRC1 locus described herein as being of interest, e.g., between and including SNPs rs602633 and rs657420. In some embodiments, the methods described herein include determining the sequence of the CELSR2 3′UTR described herein as being of interest, e.g., between and including SNPs rs12740374 and rs629301. In some embodiments, the methods described herein include determining the sequence of the PSRC1 5′UTR described herein as being of interest, e.g., between and including SNPs rs602633 and rs599839. In some embodiments, the methods described herein include determining the sequence of the entire region of the SORT1 locus described herein as being of interest. In some embodiments, the sequence is determined on both strands of DNA.

In order to detect polymorphisms and/or polymorphic variants, it will frequently be desirable to amplify a portion of genomic DNA (gDNA) encompassing the polymorphic site. Such regions can be amplified and isolated by PCR using oligonucleotide primers designed based on genomic and/or cDNA sequences that flank the site. PCR refers to procedures in which target nucleic acid (e.g., genomic DNA) is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. See e.g., PCR Primer: A Laboratory Manual, Dieffenbach and Dveksler, (Eds.); McPherson et al., PCR Basics: From Background to Bench (Springer Verlag, 2000); Mattila et al., Nucleic Acids Res., 19: 4967 (1991); Eckert et al., PCR Methods and Applications, 1: 17 (1991); PCR (eds. McPherson et al., IRL Press, Oxford); and U.S. Pat. No. 4,683,202. Other amplification methods that may be employed include the ligase chain reaction (LCR) (Wu and Wallace, Genomics 4: 560 (1989), Landegren et al., Science 241: 1077 (1988), transcription amplification (Kwoh et al., Proc. Natl. Acad. Sci. USA 86: 1173 (1989)), self-sustained sequence replication (Guatelli et al., Proc. Nat. Acad. Sci. USA 87: 1874 (1990)), and nucleic acid based sequence amplification (NASBA). Guidelines for selecting primers for PCR amplification are well known in the art. See, e.g., McPherson et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

In some cases, PCR conditions and primers can be developed that amplify a product only when the variant allele is present or only when the wild type allele is present (MSPCR or allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction.

Real-time quantitative PCR can also be used to determine copy number. Quantitative PCR permits both detection and quantification of specific DNA sequence in a sample as an absolute number of copies or as a relative amount when normalized to DNA input or other normalizing genes. A key feature of quantitative PCR is that the amplified DNA product is quantified in real-time as it accumulates in the reaction after each amplification cycle. Methods of quantification can include the use of fluorescent dyes that intercalate with double-stranded DNA, and modified DNA oligonucleotide probes that fluoresce when hybridized with a complementary DNA.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, e.g., Nielsen et al., Bioconjugate Chemistry, The American Chemical Society, 5: 1 (1994)). The PNA probe can be designed to specifically hybridize to a nucleic acid comprising a polymorphic variant conferring susceptibility to myocardial infarction.

In some cases, allele-specific oligonucleotides can also be used to detect the presence of a polymorphic variant. For example, polymorphic variants can be detected by performing allele-specific hybridization or allele-specific restriction digests. Allele specific hybridization is an example of a method that can be used to detect sequence variants, including complete haplotypes of a subject (e.g., a mammal such as a human). See Stoneking et al., Am. J. Hum. Genet. 48: 370-382 (1991); and Prince et al., Genome Res. 11: 152-162 (2001). An “allele-specific oligonucleotide” (also referred to herein as an “allele-specific oligonucleotide probe”) is an oligonucleotide that is specific for particular a polymorphism can be prepared using standard methods (see Ausubel et al., Current Protocols in Molecular Biology, supra). Allele-specific oligonucleotide probes typically can be approximately 10-50 base pairs, preferably approximately 15-30 base pairs, that specifically hybridizes to a nucleic acid region that contains a polymorphism. Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. In some cases, dot-blot hybridization of amplified oligonucleotides with allele-specific oligonucleotide (ASO) probes can be performed. See, for example, Saiki et al., Nature (London) 324: 163-166 (1986). In some embodiments, allele-specific restriction digest analysis can be used to detect the existence of a polymorphic variant of a polymorphism, if alternate polymorphic variants of the polymorphism result in the creation or elimination of a restriction site. Allele-specific restriction digests can be performed in the following manner. A sample containing genomic DNA is obtained from the individual and genomic DNA is isolated for analysis. For nucleotide sequence variants that introduce a restriction site, restriction digest with the particular restriction enzyme can differentiate the alleles. In some cases, polymerase chain reaction (PCR) can be used to amplify a region comprising the polymorphic site, and restriction fragment length polymorphism analysis is conducted (see Ausubel et al., Current Protocols in Molecular Biology, supra). The digestion pattern of the relevant DNA fragment indicates the presence or absence of a particular polymorphic variant of the polymorphism and is therefore indicative of the presence or absence of susceptibility to myocardial infarction. For sequence variants that do not alter a common restriction site, mutagenic primers can be designed that introduce a restriction site when the variant allele is present or when the wild type allele is present. For example, a portion of a nucleic acid can be amplified using the mutagenic primer and a wild type primer, followed by digest with the appropriate restriction endonuclease.

In some embodiments, fluorescence polarization template-directed dye-terminator incorporation (FP-TDI) is used to determine which of multiple polymorphic variants of a polymorphism is present in a subject (Chen et al., Genome Research 9(5): 492-498 (1999)). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

In some cases, DNA containing an amplified portion may be dot-blotted, using standard methods (see Ausubel et al., Current Protocols in Molecular Biology, supra), and the blot contacted with the oligonucleotide probe. The presence of specific hybridization of the probe to the DNA is then detected. Specific hybridization of an allele-specific oligonucleotide probe to DNA from the subject is indicative of susceptibility to myocardial infarction.

The methods can include determining the genotype of a subject with respect to both copies of the polymorphic site present in the genome. For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

Methods of nucleic acid analysis to detect polymorphisms and/or polymorphic variants can include, e.g., microarray analysis. Hybridization methods, such as Southern analysis, Northern analysis, or in situ hybridizations, can also be used (see Ausubel et al., Current Protocols in Molecular Biology, eds., John Wiley & Sons (2003)). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complementary nucleic acid sequence that selectively hybridizes to the target nucleic acid sequence. In order for a probe to hybridize to a target sequence, the hybridization probe must have sufficient identity with the target sequence, i.e., at least 70% (e.g., 80%, 90%, 95%, 98% or more) identity to the target sequence. The probe sequence must also be sufficiently long so that the probe exhibits selectivity for the target sequence over non-target sequences. For example, the probe will be at least 20 (e.g., 25, 30, 35, 50, 100, 200, 300, 400, 500, 600, 700, 800, 900 or more) nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as polymerase chain reaction (PCR), ligase chain reaction (LCR), etc., for amplification of a target sequence.

The probe can be a test probe such as a probe that can be used to detect polymorphisms as described herein. For example, the probe can hybridize to exemplary SNPs rs12740374, rs7528419, rs660240, rs629301, rs646776, rs602633, rs599839. Other exemplary SNPs useful for probes can include rs4970833, rs653635, rs6689614, rs6657811, rs608196, rs2281894, rs17035630, rs17035665, rs4970834, rs611917, rs658435, rs17035949, rs10410, rs14000, rs657420, rs672569, rs11102967, rs3832016, rs3902354, rs583104, rs4970837, and rs1277930. In some embodiments, the probe can bind to another marker sequence associated with myocardial infarction, atherosclerosis, and/or hypercholesterolemia as described herein.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially such from Applied Biosystems, e.g., the Assays-on-Demand SNP kits. Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via PCR. See, for example, Nath and Johnson, Biotechnic. Histochem. 73(1): 6-22 (1998); Wheeless et al., Cytometry 17: 319-326 (1994); and U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), TEXAS RED™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

In another aspect, the invention features arrays that include a substrate having a plurality of addressable areas, and methods of using them. At least one area of the plurality includes a nucleic acid probe that binds specifically to a sequence comprising a polymorphism described herein, and can be used to detect the absence or presence of said polymorphism, e.g., one or more SNPs, microsatellites, minisatellites, or indels, as described herein, to determine a haplotype. For example, the array can include one or more nucleic acid probes that can be used to detect a polymorphism described herein. In some embodiments, the array further includes at least one area that includes a nucleic acid probe that can be used to specifically detect another marker associated with myocardial infarction, atherosclerosis, and/or hypercholesterolemia, as described herein. In some embodiments, the probes are nucleic acid capture probes.

Generally, microarray hybridization is performed by hybridizing a nucleic acid of interest (e.g., a nucleic acid encompassing a polymorphic site) with the array and detecting hybridization using nucleic acid probes. In some cases, the nucleic acid of interest is amplified prior to hybridization. Hybridization and detecting are generally carried out according to standard methods. See, e.g., Published PCT Application Nos. WO 92/10092 and WO 95/11995, and U.S. Pat. No. 5,424,186. For example, the array can be scanned to determine the position on the array to which the nucleic acid hybridizes. The hybridization data obtained from the scan is typically in the form of fluorescence intensities as a function of location on the array.

Arrays can be formed on substrates fabricated with materials such as paper, glass, plastic (e.g., polypropylene, nylon, or polystyrene), polyacrylamide, nitrocellulose, silicon, optical fiber, or any other suitable solid or semisolid support, and can be configured in a planar (e.g., glass plates, silicon chips) or three dimensional (e.g., pins, fibers, beads, particles, microtiter wells, capillaries) configuration. Methods for generating arrays are known in the art and include, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145). The array typically includes oligonucleotide probes capable of specifically hybridizing to different polymorphic variants. Oligonucleotide probes forming an array may be attached to a substrate by any number of techniques, including, without limitation, (i) in situ synthesis (e.g., high-density oligonucleotide arrays) using photolithographic techniques; (ii) spotting/printing at medium to low density on glass, nylon or nitrocellulose; (iii) by masking, and (iv) by dot-blotting on a nylon or nitrocellulose hybridization membrane. Oligonucleotides also can be non-covalently immobilized on a substrate by hybridization to anchors, by means of magnetic beads, or in a fluid phase such as in microtiter wells or capillaries.

Arrays can include multiple detection blocks (i.e., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments. General descriptions of using oligonucleotide arrays for detection of polymorphisms can be found, for example, in U.S. Pat. Nos. 5,858,659 and 5,837,832. In addition to oligonucleotide arrays, cDNA arrays may be used similarly in certain embodiments of the invention.

The methods described herein can include providing an array as described herein; contacting the array with a sample (e.g., a portion of genomic DNA that includes at least a portion of human chromosome 1p13 (e.g., a region between SNPs rs4970833 and rs672569, a region between SNPs rs12740374 and rs599839, or a region between SNPs rs7528419 and rs646776) and/or optionally, a different portion of genomic DNA (e.g., a portion that includes a different portion of a human chromosome (e.g., including another region associated with myocardial infarction, atherosclerosis, and/or hypercholesterolemia)), and detecting binding of a nucleic acid from the sample to the array. Optionally, the method includes amplifying nucleic acid from the sample, e.g., genomic DNA that includes a portion of a human chromosome described herein, and, optionally, a region that includes another region associated with myocardial infarction, atherosclerosis, and/or hypercholesterolemia, prior to or during contact with the array.

In some aspects, the methods described herein can include using an array that can ascertain differential expression patterns or copy numbers of one or more genes in samples from normal and affected individuals (see, e.g., Redon et al., Nature 444(7118): 444-54 (2006)). For example, arrays of probes to a marker described herein can be used to measure polymorphisms between DNA from a subject having myocardial infarction, atherosclerosis, and/or hypercholesterolemia, and control DNA, e.g., DNA obtained from an individual that does not have myocardial infarction, atherosclerosis, and/or hypercholesterolemia, and has no risk factors for myocardial infarction, atherosclerosis, and/or hypercholesterolemia. Since the clones on the array contain sequence tags, their positions on the array are accurately known relative to the genomic sequence. Different hybridization patterns between DNA from an individual afflicted with myocardial infarction, atherosclerosis, and/or hypercholesterolemia and DNA from a normal individual at areas in the array corresponding to markers in human chromosome 1p13 as described herein, and, optionally, one or more other regions associated with myocardial infarction, atherosclerosis, and/or hypercholesterolemia, are indicative of a risk of such cardiovascular diseases. Methods for array production, hybridization, and analysis are described, e.g., in Snijders et al., Nat. Genet. 29: 263-264 (2001); Klein et al., Proc. Natl Acad. Sci. USA 96: 4494-4499 (1999); Albertson et al., Breast Cancer Res. and Treatment 78: 289-298 (2003); and Snijders et al. “BAC microarray based comparative genomic hybridization,” in Zhao et al. (eds), Bacterial Artificial Chromosomes: Methods and Protocols, Methods in Molecular Biology, Humana Press, 2002.

In another aspect, the invention features methods of determining the absence or presence of a haplotype associated with LDL-C levels and/or risk of myocardial infarction as described herein, using an array described above. The methods include providing a two dimensional array having a plurality of addresses, each address of the plurality being positionally distinguishable from each other address of the plurality having a unique nucleic acid capture probe. In some embodiments, the second and third samples are from first or second-degree relatives of the test subject. Binding, e.g., in the case of a nucleic acid hybridization, with a capture probe at an address of the plurality, can be detected by any method known in the art, e.g., by detection of a signal generated from a label attached to the nucleic acid.

### Methods of Determining Treatment Regimens

Described herein are a variety of methods for selecting and optimizing (and optionally administering) a treatment for a subject having or at risk of having a cardiovascular disease (e.g., myocardial infarction, atherosclerosis, and/or hypercholesterolemia) based on the presence or absence of a haplotype. The methods provided herein can include selecting a treatment regimen for a subject determined to be at risk for developing myocardial infarction, atherosclerosis, and/or hypercholesterolemia, based upon the absence or presence of a haplotype associated with myocardial infarction, atherosclerosis, and/or hypercholesterolemia as described herein. For example, the determination of a treatment regimen can also be based upon the absence or presence of other risk factors associated with cardiovascular disease as known in the art and described herein. Therefore, the methods of the invention can include selecting a treatment regimen for a subject having one or more risk factors for myocardial infarction, atherosclerosis, and/or hypercholesterolemia, and having a haplotype described herein. The methods also can include administering a treatment regimen to a subject having, or at risk for developing, myocardial infarction, atherosclerosis, and/or hypercholesterolemia to thereby treat, prevent, or delay further progression of the disease. A treatment regimen can include the administration of hypolipidemic (cholesterol lowering) drugs and/or beta-blockers to a subject identified as at risk of developing myocardial infarction, atherosclerosis, and/or hypercholesterolemia before the onset of any adverse event.

As used herein, the term “treat” or “treatment” is defined as the application or administration of a treatment regimen, e.g., a therapeutic agent or modality, to a subject, e.g., a patient. The subject can be a patient having myocardial infarction, a symptom of myocardial infarction or at risk of developing (i.e., a predisposition toward) myocardial infarction. The treatment can be to cure, heal, alleviate, relieve, alter, remedy, ameliorate, palliate, improve or affect cardiovascular disease or myocardial infarction. For example, a standard treatment regimen for myocardial infarction can include administering anticoagulant or vasodilating compounds, administering sublingual glyceryl trinitrate (nitroglycerin), and/or administering pain relief. Preventative therapeutic measures can additionally or alternatively include administering hypolipidemic medications, promoting diet and exercise, and promoting weight loss. A standard treatment regimen for atherosclerosis can include administering anticoagulant or vasodilating compounds, administering hypolipidemic medications, performing balloon angioplasty, or performing artery bypass surgery. Standard therapeutic strategies for hypercholesterolemia include administering hypolipidemic medications, promoting diet and exercise, and promoting weight loss.

The methods can be used, for example, to choose between alternative treatments (e.g., a particular dosage, mode of delivery, time of delivery) based on the risk assessment. In some embodiments, treatment for a subject having or at risk for developing myocardial infarction, atherosclerosis, and/or hypercholesterolemia is selected based on the subject's haplotype, and the treatment is administered to the subject. In some embodiments, various treatments or combinations of treatments can be administered based on the presence in a subject of a haplotype as described herein. Various treatment regimens are known for treating cardiovascular diseases including, for example, regimens as described herein.

In some cases, methods of determining a treatment regimen and/or methods of treating or preventing myocardial infarction, atherosclerosis, and/or hypercholesterolemia can further include the step of monitoring the subject, e.g., for a change (e.g., an increase or decrease) in one or more of the diagnostic criteria for cardiovascular diseases identified herein, or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same or a different therapeutic agent or modality. Generally, a decrease in one or more of the parameters described above is indicative of the improved condition of the subject, although with red blood cell and platelet levels, an increase can be associated with the improved condition of the subject.

### Using Haplotype Information

Also included herein are compositions and methods for identifying and treating subjects who have an increased risk of myocardial infarction, atherosclerosis, and/or hypercholesterolemia, such that a theranostic approach can be taken to test such individuals to determine the effectiveness of a particular therapeutic intervention (e.g., a pharmaceutical or non-pharmaceutical intervention as described herein) and to alter the intervention to (1) reduce the risk of developing adverse outcomes and (2) enhance the effectiveness of the intervention. Thus, in addition to diagnosing or confirming the predisposition to myocardial infarction, atherosclerosis, and/or hypercholesterolemia, the methods and compositions described herein also provide a means of optimizing the treatment of a subject having such a disorder. Provided herein is a theranostic approach to treating and preventing myocardial infarction, atherosclerosis, and/or hypercholesterolemia, by integrating diagnostics and therapeutics to improve the real-time treatment of a subject. Practically, this means creating tests that can identify which patients are most suited to a particular therapy, and providing feedback on how well a drug or non-pharmaceutical treatment is working to optimize treatment regimens.

Within the clinical trial setting, a theranostic method or composition of the invention can provide key information to optimize trial design, monitor efficacy, and enhance drug safety. For instance, “trial design” theranostics can be used for patient stratification, determination of patient eligibility (inclusion/exclusion), creation of homogeneous treatment groups, and selection of patient samples that are representative of the general population. Such theranostic tests can therefore provide the means for patient efficacy enrichment, thereby minimizing the number of individuals needed for trial recruitment. “Efficacy” theranostics are useful for monitoring therapy and assessing efficacy criteria. Finally, “safety” theranostics can be used to prevent adverse drug reactions or avoid medication error.

The methods described herein can include retrospective analysis of clinical trial data as well, both at the subject level and for the entire trial, to detect correlations between a haplotype as described herein and any measurable or quantifiable parameter relating to the outcome of the treatment, e.g., efficacy (the results of which may be binary (i.e., yes and no) as well as along a continuum), side-effect profile (e.g., weight gain, metabolic dysfunction, lipid dysfunction, movement disorders, or extrapyramidal symptoms), treatment maintenance and discontinuation rates, return to work status, hospitalizations, total healthcare cost, response to non-pharmacological treatments, and/or dose response curves. The results of these correlations can then be used to influence decision-making, e.g., regarding treatment or therapeutic strategies, provision of services, and/or payment. For example, a correlation between a positive outcome parameter (e.g., high efficacy, low side effect profile, high treatment maintenance/low discontinuation rates, good return to work status, low hospitalizations, low total healthcare cost, favorable response to non-pharmacological treatments, and/or acceptable dose response curves) and a selected haplotype can influence treatment such that the treatment is recommended or selected for a subject having the selected haplotype.

This document also provides methods and materials to assist medical or research professionals in determining whether a particular treatment regimen is optimal. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether specific polymorphic variants are present in a biological sample from a subject, and (2) communicating information about polymorphic variants to that professional.

After information about specific polymorphic variants is reported, a medical professional can take one or more actions that can affect patient care. For example, a medical professional can record information in the patient's medical record regarding the patient's likely response to a given treatment for myocardial infarction, atherosclerosis, and/or hypercholesterolemia. In some cases, a medical professional can record information regarding a treatment assessment, or otherwise transform the patient's medical record, to reflect the patient's current treatment and response haplotype. In some cases, a medical professional can review and evaluate a patient's entire medical record and assess multiple treatment strategies for clinical intervention of a patient's condition.

A medical professional can initiate or modify treatment after receiving information regarding a patient's haplotype, for example. In some cases, a medical professional can recommend a change in therapy based on the subject's haplotype. In some cases, a medical professional can enroll a patient in a clinical trial for, by way of example, detecting correlations between a haplotype as described herein and any measurable or quantifiable parameter relating to the outcome of the treatment as described above.

A medical professional can communicate information regarding a patient's expected response to a treatment to a patient or a patient's family. In some cases, a medical professional can provide a patient and/or a patient's family with information regarding myocardial infarction, atherosclerosis, or hypercholesterolemia and response assessment information, including treatment options, prognosis, and referrals to specialists. In some cases, a medical professional can provide a copy of a patient's medical records to a specialist.

Any appropriate method can be used to communicate information to another person (e.g., a professional). For example, information can be given directly or indirectly to a professional. For example, a laboratory technician can input a patient's polymorphic variant haplotype as described herein into a computer-based record. In some cases, information is communicated by making a physical alteration to medical or research records. For example, a medical professional can make a permanent notation or flag a medical record for communicating the response haplotype determination to other medical professionals reviewing the record. In addition, any type of communication can be used to communicate haplotype and/or treatment information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

### Articles of Manufacture

Also within the scope of the invention are articles of manufacture comprising a probe that hybridizes with a region of human chromosome as described herein and can be used to detect a polymorphism described herein. For example, any of the probes for detecting polymorphisms described herein can be combined with packaging material to generate articles of manufacture or kits. The kit can include one or more other elements including: instructions for use; and other reagents such as a label or an agent useful for attaching a label to the probe. Instructions for use can include instructions for diagnostic applications of the probe for assessing risk of myocardial infarction, atherosclerosis, and/or hypercholesterolemia in a method described herein. Other instructions can include instructions for attaching a label to the probe, instructions for performing in situ analysis with the probe, and/or instructions for obtaining a sample to be analyzed from a subject. In some cases, the kit can include a labeled probe that hybridizes to a region of human chromosome as described herein.

The kit can also include one or more additional probes that hybridize to the same chromosome (e.g., chromosome 1p13) or another chromosome or portion thereof that can have an abnormality associated with risk for myocardial infarction, atherosclerosis, and/or hypercholesterolemia. For example, the additional probe or probes can be: a probe that hybridizes to human chromosome 1p13 or a portion thereof (e.g., a probe that detects a sequence associated with myocardial infarction, atherosclerosis, and/or hypercholesterolemia in this region of chromosome 1). A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes.

Kits for use in self-testing can also be provided. Such test kits can include devices and instructions that a subject can use to obtain a biological sample (e.g., buccal cells, blood) without the aid of a health care provider. For example, buccal cells can be obtained using a buccal swab or brush, or using mouthwash.

Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

The invention will be further described in the following examples, which do not limit the scope of the invention described in the claims.

### EXAMPLES

### Example 1

### Association of Chromosome 1p13 Locus with Very Small LDL Particles

LDL-C comprises a variety of lipoprotein particles ranging in size and density. To determine whether the 1p13 locus selectively affects certain LDL subclasses, a recently-developed ion mobility (IM) technique was used to measure lipoprotein subclasses in more than 4,500 individuals in a longitudinal community-based population cohort, the Malmö Diet and Cancer Study-Cardiovascular Cohort (MDC-CC) in Malmö, Sweden. The MDC-CC is a prospective, community-based epidemiological cohort of 6,103 residents of Malmö, Sweden, for whom a comprehensive analysis of cardiovascular risk factors has been performed. The IM technique provides a comprehensive profile of plasma lipoprotein particles including HDL, LDL, intermediate-density lipoprotein (IDL), and very-low-density lipoprotein (VLDL) particles. IM can also separately measure the four LDL particle subclasses (large, medium, small, and very small), the last of which is further subdivided into subfractions 3B, 4A, 4B, and 4C.

The ion mobility (IM) method of lipoprotein measurement was applied to archived baseline blood samples from these individuals to directly quantify the full spectrum of lipoprotein particles. The 1p13 SNP rs646776 , a variant of which has been shown to be strongly associated with LDL-C, was genotyped as previously described (Musunuru et al., Arterioscler. Thromb. Vasc. Biol., 29(11): 1975-80 (2009)). Multivariable linear regression analyses were used to test whether each of the lipid or lipoprotein measures differed according to an increasing copy number of the SNP minor allele, adjusted for age, gender, and diabetes status. SPSS (version 16.0) was used for the analyses. Mean plasma lipid and lipoprotein levels were calculated for individuals with zero, one, or two minor alleles of rs646776 in MDC-CC. All values are in units of nmol/L except LDL-C, which is mmol/L. Ratios of major allele homozygotes to minor allele homozygotes were normalized to the mean level in minor allele homozygotes. P values derived from logistic regression analyses were adjusted for sex, age, and diabetes status.

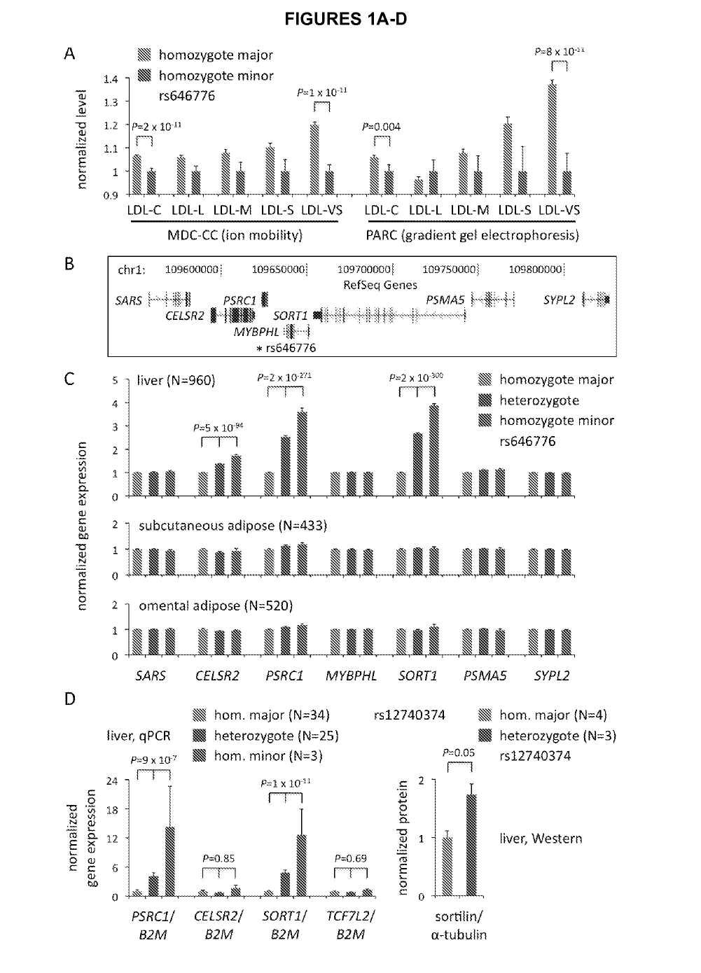

After adjusting for age, gender, and diabetes status, it was observed that an index SNP in the 1p13 locus, rs646776, was most highly associated with the very small LDL (LDL-VS) lipoprotein subclass (P=1.1×10⁻¹¹) (FIG. 1A). Homozygotes for the major allele of rs646776 (AA) had a 20% greater concentration of plasma LDL-VS particles than homozygotes for the minor allele (GG) of the SNP (FIG. 1A; Table 1). Smaller changes were seen with the other lipoprotein subclasses, as well as the total LDL particle concentration (LDL-P) and LDL-C. Among the LDL-VS subfractions (LDL-4A, LDL-4B, and LDL-4C), the greatest change was seen with LDL-4B (P=1.1×10⁻¹⁷, 27% change) (Table 1).

Similar analyses were performed in a second study, the Pharmacogenomics and Risk of Cardiovascular Disease study (PARC), in which lipoproteins were measured by a different technique, gradient gel electrophoresis, and similar results to those in MDC-CC were observed (FIG. 1A; Table 2).

TABLE 1
Malmö Diet and Cancer Study - Cardiovascular Cohort

| Minor | | | | | | | | | | | | | |
|---|---|---|---|---|---|---|---|---|---|---|---|---|---|
| alleles | N | HDL-S | HDL-L | LDL-VS | LDL-S | LDL-M | LDL-L | IDL-S | IDL-L | VLDL-S | VLDL-M | LDL-L | LDL-C |
| 0 | 2689 | 2972 | 1633 | 114 | 82.3 | 126 | 441 | 121 | 216 | 54.3 | 36.7 | 9.47 | 4.23 |
| 1 | 1607 | 2913 | 1624 | 104 | 79.1 | 121 | 424 | 116 | 213 | 52.3 | 35.6 | 9.18 | 4.08 |
| 2 | 279 | 3067 | 1690 | 94.8 | 74.7 | 116 | 416 | 115 | 213 | 53.2 | 35.7 | 9.05 | 3.97 |
| Ratio | | 0.97 | 0.97 | 1.20 | 1.10 | 1.09 | 1.06 | 1.05 | 1.01 | 1.02 | 1.03 | 1.05 | 1.07 |
| P value | | 0.84 | 0.75 | 1.1 × 10⁻¹¹ | 0.03 | 0.02 | .0004 | .0002 | 0.24 | 0.006 | 0.02 | 0.04 | 2.4 × 10⁻¹¹ |

TABLE 2
Pharmacogenomics and Risk of Cardiovascular Disease study

| Minor | | | | | | |
|---|---|---|---|---|---|---|
| alleles | N | LDL-VS | LDL-S | LDL-M | LDL-L | LDL-C |
| 0 | 1196 | 14.4 | 17.1 | 26.4 | 56.3 | 3.45 |
| 1 | 589 | 12.5 | 16.7 | 26.1 | 54.5 | 3.35 |
| 2 | 75 | 10.5 | 14.2 | 24.5 | 58.4 | 3.26 |
| Ratio | | 1.37 | 1.20 | 1.08 | 0.96 | 1.06 |
| P value | | 8 × 10⁻¹¹ | 0.16 | 0.48 | 0.29 | 0.004 |

### Example 2

### The 1p13 Locus Regulates Expression of Several Genes Specifically in Human Liver

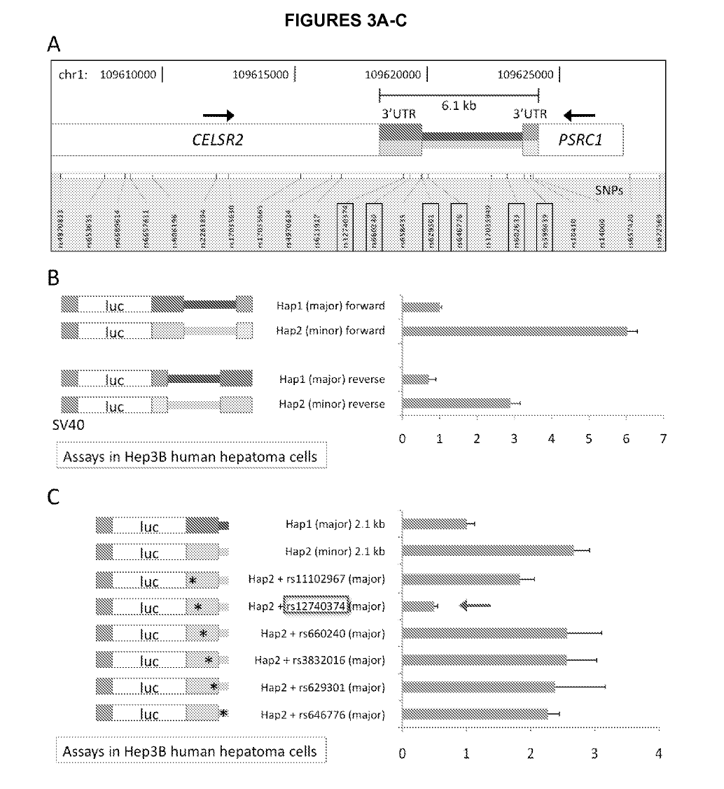

The SNPs in the 1p13 locus reported to be most highly associated with LDL-C (rs646776, rs599839, and rs12740374) are in very high linkage disequilibrium in populations of European descent (r² =1 in International HapMap Project CEU subjects) and form a distinct haplotype block. See Kathiresan et al., Nat. Genet. 40: 189-197 (2008); Kathiresan et al., Nat. Genet. 41: 56-65 (2009). They lie in a noncoding DNA region between two genes, CELSR2 (cadherin EGF LAG seven-pass G-type receptor 2) and PSRC1 (proline/serine-rich coiled-coil 1) (FIGS. 1B, 3A). Expression quantitative trait locus (eQTL) analyses were previously attempted to explore whether 1p13 SNPs are cis-acting regulators of nearby genes, with the reasoning that a noncoding DNA variant may affect transcription of the causal gene. See Kathiresan et al., Nat. Genet. 40: 189-197 (2008); Kathiresan et al., Nat. Genet. 41: 56-65 (2009). Those studies have been extended by mRNA expression of genes in or near the 1p13 locus in three types of human tissue samples: liver, subcutaneous fat, and omental fat. To evaluate whether SNPs serve as eQTLs with putative cis regulatory effects on liver and adipose gene expression traits, 782,476 SNPs were genotyped and expression levels of 39,280 transcripts profiled in 960 human liver samples, 433 human subcutaneous adipose samples, and 520 human omental adipose samples. Tissue samples were either postmortem or surgical resections from organ donors or elective cases. Methods for tissue collection, RNA and DNA isolation, expression profiling, and DNA genotyping were performed as previously described by, for example, Schadt et al., PLoS Biol. 6: e107 (2008). The correlations of rs646776 with all transcripts within 200 kb upstream or downstream of the SNP position were studied.

In liver, homozygosity for the minor allele of rs646776 was highly associated (P<0.001) with elevated transcript levels of four genes: CELSR2, PSRC1, SORT1 (sortilin), and PSMA5 (proteasome (prosome, macropain) subunit, alpha type, 5) (FIG. 1C). Other nearby genes, such as SARS (seryl-tRNA synthetase), MYBPHL (myosin binding protein H-like), and SYPL2 (synaptophysin-like 2), did not display significant changes in transcript level. SORT1 displayed the greatest degree of change, with homozygotes for the minor alleles having a 3.9-fold increase in SORT1 expression compared to homozygotes for the major allele. PSRC1 displayed a 3.6-fold change and CELSR2 a 1.7-fold change in expression. The expression levels of the three genes were highly correlated, with Spearman rho coefficients ˜0.6 in each gene-by-gene comparison, suggesting transcriptional coregulation by a shared mechanism.

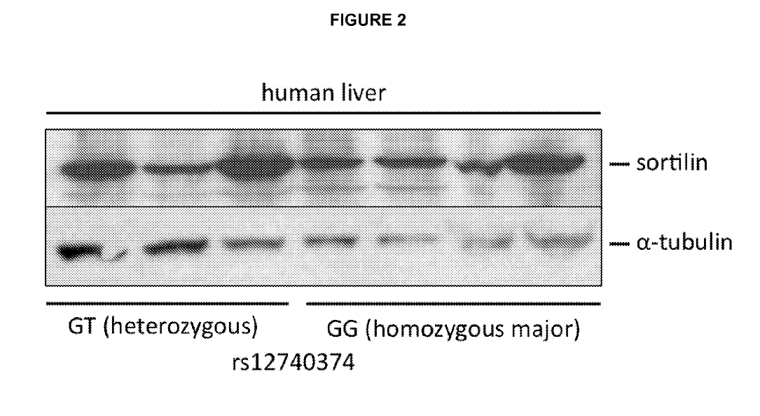

The liver eQTL assays were replicated in an independent cohort of 62 human liver samples, from which rs12740374 was directly genotyped and SORT1, PSRC1, and CELSR2 expression were measured with quantitative reverse transcriptase polymerase chain reaction (qRT-PCR). Homozygotes for the minor allele displayed more than 12-fold higher SORT1 and PSRC1 expression than homozygotes for the major allele; there was no significant change for CELSR2 (FIG. 1D). Western blot analysis of a limited number of lysates from these liver samples demonstrated a significant increase in sortilin protein abundance in heterozygotes compared to major allele homozygotes (FIG. 1D, FIG. 2).

Notably, all of the gene expression changes in liver were not seen in either of the two adipose tissue types (FIG. 1C), suggesting that the regulatory mechanism underlying the altered gene expression is liver-specific.

### Example 3

### Noncoding DNA Variant Accounts for Expression Changes in the 1p13 Locus

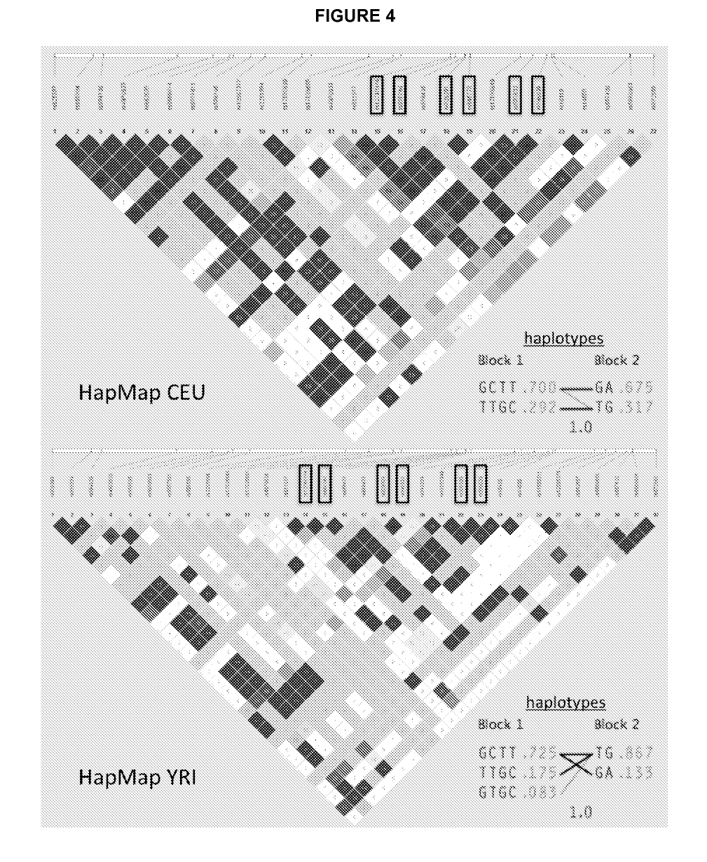

Fine mapping of the 1p13 locus was performed in order to define the minimal DNA region responsible for the observed gene expression and lipoprotein associations. Because rs646776, rs599839, and rs12740374 lie between the CELSR2 and PSRC1 genes, other SNPs were identified in the International HapMap Project database spanning the two genes and association analyses were performed with LDL-C using data from a recent genome-wide association meta-analysis of individuals of European descent. See Kathiresan et al., Nat. Genet. 41: 56-65 (2009). Out of 19 other SNPs, three SNPs with P values comparable to rs646776, rs599839, and rs12740374 (P values ranging from 1.8×10⁻⁴² to 8.3×10⁻⁴¹) were identified: rs660240, rs629301, and rs602633 (Table 3; six presented in bold text). No SNPs having P values lower than rs646776, rs599839, and rs12740374 were identified (Table 3). The six SNPs (including rs646776, rs599839, and rs12740374) cluster in a noncoding DNA region that is 6.1 kb in size, spanning the 3′ untranslated region (3′UTR) of CELSR2, the intergenic region, and the PSRC1 3′UTR oriented in the opposite direction (FIG. 3A). Based on minor allele frequencies and haplotype data available from the International HapMap Project, these six SNPs are in high linkage disequilibrium (FIG. 4) and comprise two predominant haplotypes in individuals of European descent, with the “major” haplotype present on 68% of European chromosomes 1 and the “minor” haplotype on 29% of chromosomes 1 (shown as Hap1 and Hap2 in Table 4, respectively).

Two human bacterial artificial chromosomes (BACs) harboring the major and minor haplotypes of the 6.1 kb noncoding DNA region were identified. Regions on the two BACs were sequenced in full. As shown in Table 4, sixteen polymorphisms were identified, seven of which were previously cataloged in HapMap, six in the dbSNP database but not in HapMap, and three novel polymorphisms. Thirteen of the variants were SNPs, with the other three being single-base insertion-deletion variants (Table 4).

TABLE 3
Associations of Polymorphisms with LDL-C

| | | | Allele | P value | P value |
|---|---|---|---|---|---|
| | | | associated | for | for |
| | | SEQ | with | ~20,000 | ~9,000 |
| | Polymorphisms associated with | ID | increased | European | African |
| SNP | increased LDL-C | NO: | LDL-C | Americans | Americans |
| rs4970833 | GCATTTCTGGCTGAGAGGAAGGGAGC [A/G] | 1 | A | 4.2 × 10⁻¹¹ | 0.12 |
| | GCTGGGAAGGTGCCACCTTGCTGGG | | | | |
| rs653635 | GCGCGTGCGGAACATGAGGCTGAGGT [A/G] | 2 | G | 0.12 | |
| | CCAGGGTTGGGAGATGGGCAGCGAG | | | | |
| rs6689614 | CAGGGTGTGCGGGTGAGCGATACGCC [A/G] | 3 | A | 4.6 × 10⁻¹¹ | 0.12 |
| | GAGGGGGTTAACAGCCTGGATCCCA | | | | |
| rs6657811 | TAAGGATCCAGGGCAACGGGCAGGTT [A/T] | 4 | A | 3.3 × 10⁻²² | 3.3 × 10⁻⁷ |
| | TCAGGTGCCTGGGGCCACATGCTGG | | | | |
| rs608196 | GGCCAGAGCTCATGGGTTACACATTG [A/G] | 5 | A | 0.098 | |
| | CCCAGGGTCCCCCTGCGCCTCAGAC | | | | |
| rs2281894 | TCCCAGCAGCTAGCCCTGCTCCTGCG [A/C] | 6 | C | 7.2 × 10⁻⁵ | 0.08 |
| | AACGCCACGCAGCACACAGCTGGCT | | | | |
| rs17035630 | TGTGTGTGGGTCCAGGCACAGGGCTG [A/G] | 7 | A | 0.0066 | 0.94 |
| | GAAGCTCTTATGTAGAGAATGAGGA | | | | |
| rs17035665 | TGTGGCCCTCCTTTGCTGTCCTCCTG [C/T] | 8 | C | 0.00047 | |
| | TGCTGGGGCCGCGTGTCCTCAGGAC | | | | |
| rs4970834 | CAGCCATCCCACTCCCCACTTACTGA [C/T] | 9 | C | 1.6 × 10⁻²⁵ | 0.002 |
| | CTCTCTGTTCCCTGCCTAGTCCTAC | | | | |
| rs611917 | TCAGTGCGGACTCCTCCCTAGGAAGA [C/T] | 10 | T | 8.9 × 10⁻²⁹ | 9.2 × 10⁻¹⁵ |
| | GAGACCATGCTGGAGGGGTCAGCCT | | | | |
| rs12740374 | ACAGTGCTGGCTCGGCTGCCCTGAGG [G/T] | 11 | G | 1.8 × 10⁻⁴² | 2.3 × 10⁻²⁰ |
| | TGCTCAATCAAGCACAGGTTTCAAG | | | | |
| rs660240 | GGGACTGGACACGAACCCATCCCAAC [A/G] | 12 | G | 8.3 × 10⁻⁴¹ | |
| | CAAAAAGCAAATAAATAAAACAAAT | | | | |
| rs658435 | CAACAACCAAACTGTACAATCTGATG [A/G] | 13 | A | 0.023 | 0.18 |
| | TTAGTACCAAGTTAGCATCCAGCAT | | | | |
| rs629301 | AAAAACAACAACAACAAAACGCTACC [A/C] | 14 | A | 2.2 × 10⁻⁴¹ | |
| | TATTTACAGCAACAACCAAACTGTA | | | | |
| rs646776 | CTATTTGGGAGCAGTGTCATGGACAT [A/G] | 15 | A | 2.2 × 10⁻⁴¹ | 1.6 × 10⁻¹³ |
| | GGCAGAGGGACAGGCTTATCAGCCA | | | | |
| rs17035949 | CTACACCAAATCTGTTAAACGTGTCT [G/T] | 16 | T | 1.0 × 10⁻⁶ | 0.02 |
| | TGTTATTCCTTCAACAAACACCATC | | | | |
| rs602633 | CCCAGCCCCCTGCTCCAATTTCTAAC [A/C] | 17 | C | 7.6 × 10⁻⁴¹ | 0.05 |
| | AATTTGGAGTAAATCTCTAATTCCA | | | | |
| rs599839 | AAAGAGAAAGAAATAGGAGCAGGATC [A/G] | 18 | A | 7.3 × 10⁻⁴² | 0.03 |
| | ACTTCCAGATATACAGAGAATATAA | | | | |
| rs10410 | TGGCATTGGTTCACTGGACATTTCCA [C/T] | 19 | T | 0.057 | 0.62 |
| | GTGAGCGGCCTCCGTAGCTAACCTC | | | | |
| rs14000 | GACAAACCACTTGCCTGTACTTCTCA [C/T] | 20 | C | 0.062 | 0.86 |
| | CTTCTATTTGTTCATTTCACTGCTG | | | | |
| rs657420 | AGAACGGTTGTGCTGCCTAACGTGGC [C/T] | 21 | T | 1.3 × 10⁻⁹ | 0.003 |
| | GCTAACCGACGCTCTACGGGAGGAA | | | | |
| rs672569 | TATGGACTTGCCCTGAATTATTTCCT [A/G] | 22 | G | 2.0 × 10⁻¹⁴ | |
| | TGCAAGGGCCAAGAACCCTCTCTTG | | | | |

TABLE 4
Single-Nucleotide Polymorphisms in the Major and
Minor Haplotypes

| | | Seq | | |
|---|---|---|---|---|
| | | ID | Hap1 | Hap2 |
| Variant | | No. | allele | allele |
| Novel 1- | CCACCTAAGGCCATCTAGTGCCAACT[-/C] | 23 | — | C |
| base indel | CCCCCCCCACCATTCCCCTCACTGC | | | |
| rs7528419 | CACGCAGCCAGGGCTTCACACCCTTC[A/G] | 24 | A | G |
| (HapMap) | GGCTGCACCCGGGCAGGCCTCAGAA | | | |
| rs11102967 | GGTGAGGGGCCAGGGCAAAGGGTGTG[C/T] | 25 | T | C |
| (dbSNP) | CTCGTCCTGCCCGCACTGCCTCTCC | | | |
| rs12740374 | ACAGTGCTGGCTCGGCTGCCCTGAGG[G/T] | 26 | G | T |
| (HapMap) | TGCTCAATCAAGCACAGGTTTCAAG | | | |
| rs660240 | GGGACTGGACACGAACCCATCCCAAC[A/G] | 17 | G | A |
| (HapMap) | CAAAAAGCAAATAAATAAAACAAAT | | | |
| rs3832016 | TGCAGGAATTGCTGAGGGGAGAAGAC[-/A] | 28 | A | — |
| (dbSNP) | GGGGGAGAATCCACGGCAGAAAAGC | | | |
| rs629301 | AAAAACAACAACAACAAAACGCTACC[A/C] | 29 | A | C |
| (HapMap) | TATTTACAGCAACAACCAAACTGTA | | | |
| rs646776 | CTATTTGGGAGCAGTGTCATGGACAT[A/G] | 30 | A | G |
| (HapMap) | GGCAGAGGGACAGGCTTATCAGCCA | | | |
| Novel SNP | CCTAGGCTCAAGTGATCCTCCCGCCT[C/T] | 31 | T | C |
| | GGCCTCCCAAACTGGGGTCACAGAT | | | |
| rs3902354 | AGCTGCGATTACAGGCATGTACCACC[G/T] | 32 | T | G |
| (dbSNP) | CACCCAGCTAATTTTTTTGTATTTT | | | |
| Novel SNP | GATCGCACCACTGCACTCCAGCCTGG[A/G] | 33 | A | G |
| | TAATAGAGCCAGACCTTGTCTCAAA | | | |
| rs583104 | AGATGAAAGTTAAGTGTAGCTTAATT[A/C] | 34 | A | C |
| (dbSNP) | ACAGTATGTGAGGCTATTAAGCTAG | | | |
| rs602633 | CCCAGCCCCCTGCTCCAATTTCTAAC[A/C] | 35 | C | A |
| (HapMap) | AATTTGGAGTAAATCTCTAATTCCA | | | |
| rs4970837 | AAAAATTAGCCGGACGTGGGTGGCAG[C/G/T] | 36 | T | G |
| (dbSNP) | CGCCTGTAATCTCAGCTACTCGGGA | | | |
| rs1277930 | ACAGAGCAAGATTCTGTCTAAAAAAA[A/G] | 37 | A | G |
| (dbSNP) | AGAAAGAAATAGGAGCAGGATCGAC | | | |
| rs599839 | AAAGAGAAAGAAATAGGAGCAGGATC[A/G] | 38 | A | G |
| (HapMap) | ACTTCCAGATATACAGAGAATATAA | | | |

The 6.1 kb region from each BAC, representing the major (Hap1) and minor (Hap2) haplotypes, was subcloned into a firefly luciferase expression construct. The region spanning precisely between the stop codon of CELSR2 and the stop codon of PSRC1 was placed just distal to the stop codon of the luciferase gene in both the “forward” orientation and the “reverse” orientation, thereby substituting luciferase in either the position of CELSR2 or the position of PSRC1 relative to the noncoding region. PCR was also used to generate smaller truncations. All constructs were verified by DNA sequencing. Each of these constructs was co-transfected with a Renilla luciferase construct into Hep3B cultured human hepatocellular carcinoma cells. The FuGene 6 transfection reagent (Roche) was used in the ratio 1 μg:100 ng:3 μL mixed with Opti-MEM I Reduced Serum Medium (Invitrogen) for a 100 μL mix, of which 20 μL was used for each well of 24-well plates. Hep3B cultured human hepatocellular carcinoma cells were chosen to replicate the in vivo characteristics of the human liver samples in which the eQTL analyses had been performed. In some assays, BLN CL.2 cultured mouse embryonic liver cells or NIH 3T3 cultured mouse fibroblast cells were transfected, each at roughly 50% confluence. In one condition, a lentivirus encoding the C/EBPα cDNA was added to the NIH 3T3 cells concomitantly with the transfection mix. Forty-eight hours after transfection, firefly and Renilla luciferase activity were measured using the Dual-Luciferase Reporter Assay System (Promega) according to the manufacturer's protocol, using untransfected cells to adjust for background activity. It was observed that in both the “forward” (CELSR2) and “reverse” (PSRC1) orientations, the minor haplotype of the noncoding region produced significantly greater luciferase expression than the major haplotype. This directionality is consistent with the human liver eQTL analyses where the minor allele is associated with higher concentration of neighboring genes (FIG. 3B; compare to FIG. 1C). These results were replicated in a second hepatic cell line, BNL CL.2 cultured mouse embryonic liver cells.

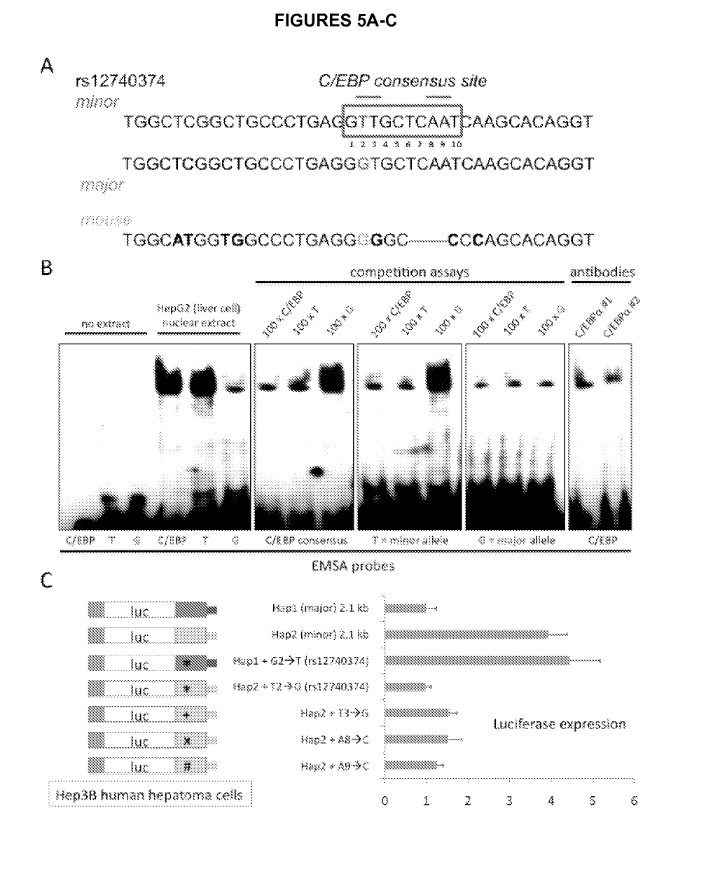

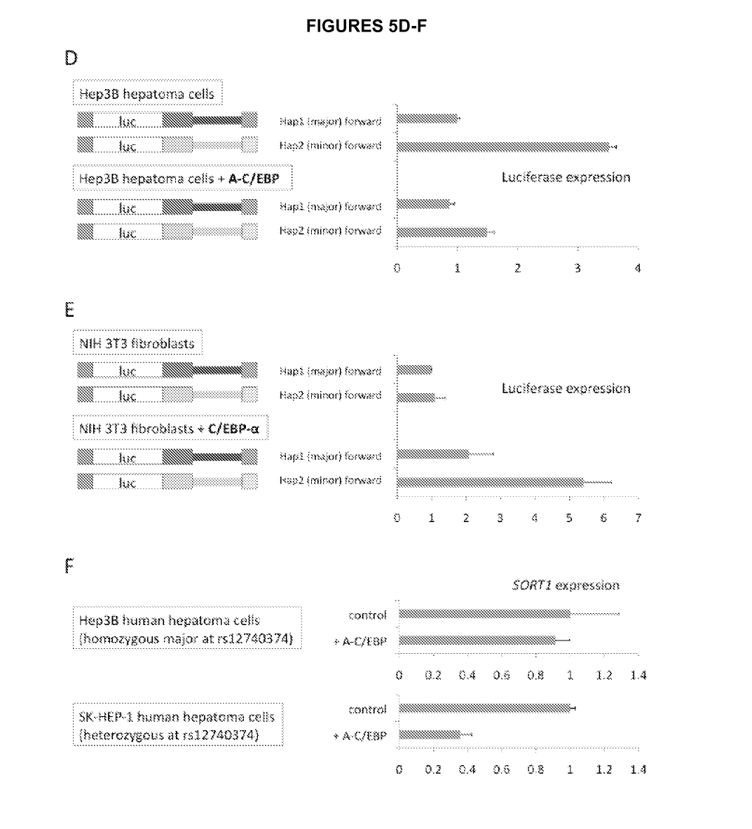

Studies with luciferase constructs harboring composites and truncations of the noncoding region in Hep3B human hematoma cells were repeated and the haplotype-specific effect was localized to a smaller 2.1 kb element spanning the CELSR2 3′UTR and the proximal portion of the intergenic region. This was followed by testing of an array of constructs in which single polymorphisms in the minor haplotype were switched to major alleles. The SNP rs12740374 was identified as being sufficient to confer the haplotype-specific effect (FIGS. 3C, 5sC).

SNP rs12740374 and 15 other SNPs in the 6.1 kb noncoding region were genotyped in 9,000 African-Americans. Whereas six SNPs were all highly associated with LDL-C in individuals of European ancestry (Table 3; presented in bold font), it was observed that, in African-Americans, rs12740374 alone had the strongest evidence for association (P=2.3×10⁻²⁰ for rs12740374 vs. 9.2×10⁻¹⁵ at the next best 1p13 SNP; Table 3). This is consistent with rs12740374 being in high linkage disequilibrium with other nearby SNPs in HapMap Europeans (CEU) but not so in HapMap Africans (YRI) (FIG. 4).

It was observed that rs12740374 altered a predicted consensus binding site for CCAAT/enhancer binding protein (C/EBP) transcription factors, with the minor allele preserving the site and the major allele disrupting the site (FIG. 5A). C/EBPα is a liver-enriched transcriptional factor that regulates the expression of numerous hepatic genes involved in a variety of metabolic processes. Luciferase constructs were tested in Hep3B hepatoma cells, replicating the haplotype-specific expression difference; infecting these cells with a lentivirus encoding a dominant-negative C/EBP protein significantly reduced the expression difference. Binding of the rs12740374 minor and major allele sequences by C/EBP was tested by electrophoretic mobility shift assay. Primers with the consensus C/EBPα binding site were previously described. See Osada et al., J. Biol. Chem. 271: 3891-3896 (1996). C/EBPα-F, 5′-CTAGGCATATTGCGCAATATGC-3′ (SEQ ID NO:39); C/EBPα-R, 5′-GCATATTGCGCAATATGCCTAG-3′ (SEQ ID NO:40). Primers for rs12740374 were designed based on genomic sequences surrounding the SNP (ncbi.nlm.nih.gov/projects/SNP/ on the World Wide Web): rs12740374_T-F, 5′-TGCCCTGAGGTTGCTCAATCA-3′ (SEQ ID NO:41); rs12740374T-R, 5′-TGATTGAGCAACCTCAGGGCA-3′ (SEQ ID NO:42); rs12740374_G-F, 5′-TGCCCTGAGGGTGCTCAATCA-3′ (SEQ ID NO:43); rs12740374G-R: 5′-TGATTGAGCACCCTCAGGGCA-3′ (SEQ ID NO:44). All primers were ordered from Invitrogen. Individual primers were labeled with a biotin 3′ end DNA labeling kit (Pierce) according to instructions, and the efficiency of labeling was tested by a dot-test that confirmed that all the primers were labeled similarly. Corresponding forward and reverse primers were annealed to create 3′ end biotin-labeled double-stranded probes. EMSA reactions were performed with the biotin 3′ end DNA labeling kit (Pierce) according to instructions, with 8 ng of nuclear extract from the hepatic cell line HepG2 per reaction (Active Motif). For competition assays, a 100-fold excess of unlabeled probe was used. To test for involvement of CEBP/α in interaction with the probes, the HepG2 nuclear extract was preincubated for 15 minutes at room temperature with either of two antibodies for CEBP/α (39306, Active Motif; 2295, Cell Signaling). The protein complexes were resolved on 6% DNA retardation gels (Invitrogen) for 1 hour at 100 V, transferred to Biodyne B Nylon Membranes (Pierce), crosslinked, and processed with the Chemiluminescent Nucleic Acid Detection Module (Pierce). It was observed that the minor allele sequence to be shifted to an equivalent degree as a consensus C/EBP binding sequence, with minimal shifting of the major allele sequence; addition of either of two C/EBPα antibodies impaired the binding (FIG. 5B).

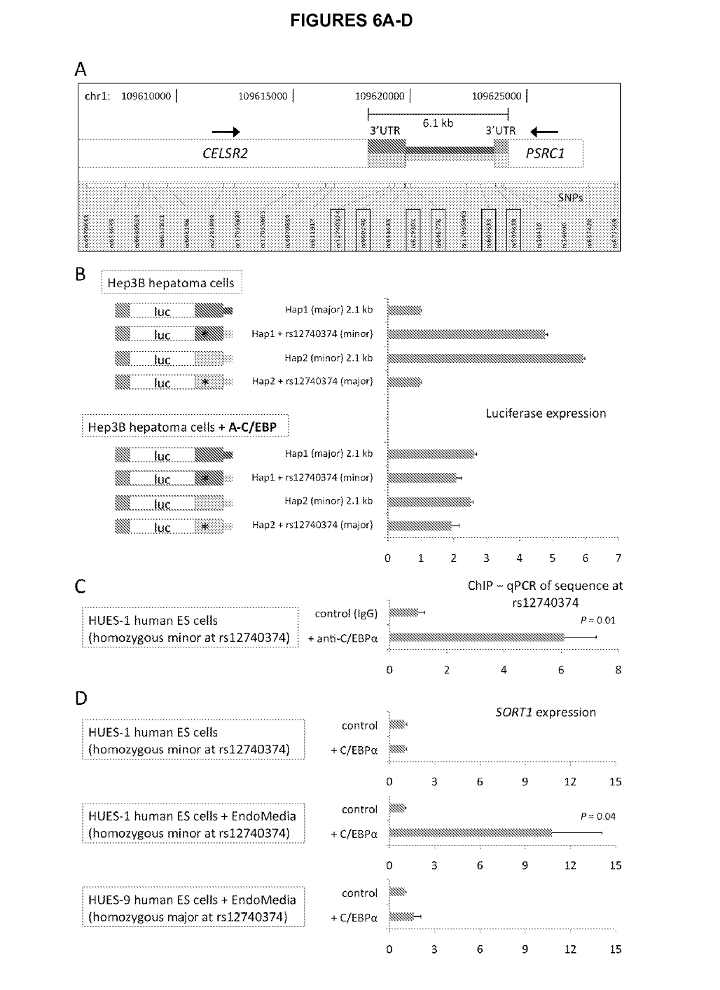

Luciferase constructs were tested in Hep3B cells infected with a lentivirus encoding a dominant negative C/EBP protein (A-C/EBP). Significantly reduced differences in haplotype-specific expression were observed (FIG. 5D, FIG. 6B). Luciferase constructs were also tested in NIH 3T3 cultured mouse fibroblast cells (FIG. 5E) and no haplotype-specific expression difference was found, suggesting a liver-specific mechanism, consistent with the eQTL analyses. Infecting 3T3 cells with a lentivirus encoding C/EBPα restored the haplotype-specific effect (FIG. 5E). Other nucleotides in the consensus binding site predicted to be critical for C/EBP protein-DNA interactions were altered and it was found that they were needed for transcriptional activation by the minor haplotype (FIG. 5C). Furthermore, it was determined that C/EBPα binds to the site of rs12740374 in homozygous minor allele cells by chromatin immunoprecipitation (FIG. 6C).

Assays were performed to determine whether C/EBP proteins can influence SORT1 expression via rs12740374. When dominant-negative A-C/EBP was added to Hep3B cells homozygous for the major allele at rs12740374, no difference in SORT1 expression was detected as measured by qRT-PCR (FIG. 5F). In contrast, when A-C/EBP was added to SK-HEP-1 cultured human hepatoma cells heterozygous (one minor allele) at rs12740374, a three-fold reduction in SORT1 expression was observed (FIG. 5F). When C/EBPα was added to human embryonic stem (ES) cells homozygous for the minor allele at rs12740374 (HUES-1), no change in SORT1 expression was observed, presumably because ES cells do not harbor cofactors of C/EBP needed for transcriptional activation of the gene (FIG. 6D). When HUES-1 cells were differentiated into endoderm, the first step towards liver differentiation, addition of C/EBPα resulted in a significant increase in SORT1 expression (FIG. 6D). In contrast, human ES cells homozygous for the major allele at rs12740374 (HUES-9), when differentiated into endoderm, showed no difference in SORT1 expression (FIG. 6D). These observations suggest that a C/EBP family member or a related protein binds to the minor haplotype at rs12740374 in vivo and is thereby involved in the haplotype-specific gene expression effects observed in human liver (see FIG. 1C).

Together, these findings demonstrate that rs12740374 is the causal variant responsible for the liver-specific association between the 1p13 locus and gene expression and, by extension, the associations with LDL-C, LDL-VS particles, and MI risk. Furthermore, these findings suggest that rs12740374 is a causal variant in all ethnic groups, not just individuals of European descent.

### Example 4

### Overexpression of Sort1 in Mouse Liver Modulates Plasma Lipid and Lipoprotein Levels

Of the genes differentially expressed in human liver by 1p13 haplotype, the SORT1 gene showed the greatest degree of change, with homozygotes for the minor alleles having a 3.9-fold increase in SORT1 expression compared to homozygotes for the major allele (FIG. 1C). SORT1 encodes the sortilin protein, also known as neurotensin receptor 3, which functions as a multiligand receptor. Sortilin harbors an N-terminal propeptide with a furin cleavage site, a Vps10 domain for ligand binding, a single transmembrane domain, and a C-terminal cytoplasmic tail that functions as an endolysosomal sorting motif. The protein localizes in the endoplasmic reticulum (ER) and Golgi apparatus and has roles in both endocytosis and intracellular trafficking of other proteins. Sortilin is implicated in the regulation of neuronal apoptosis via the formation of extracellular complexes with proneurotrophins and the p75 neurotrophic receptor. Sortilin is also highly expressed in adipose and is involved in the formation and stabilization of Glut4-containing storage vesicles for insulin-responsive glucose uptake in adipocytes and skeletal muscle. The protein was originally identified through affinity chromatography with the receptor-associated protein (RAP), also called low density lipoprotein receptor-related protein associated protein 1 (LRPAP1); sortilin has also been reported to bind and degrade lipoprotein lipase (LPL), apolipoprotein E, and apolipoprotein A-V.

Because of its biological plausibility and the magnitude of the gene expression change in human liver by 1p13 haplotype, overexpression studies of SORT1 were performed in mice. Wild-type mice have very low levels of plasma LDL-C compared to humans. Furthermore, mice preferentially produce a truncated form of apolipoprotein B (apoB-48) in liver by RNA editing of the Apob mRNA, whereas in humans only the full-length form (apoB-100) is produced in liver. Therefore, “humanized” mice that lack the Apob RNA-editing enzyme (Apobec1−/−) and that have increased plasma LDL particle levels, either by increased synthesis (human APOB transgenic; APOB Tg) or decreased clearance (LDL receptor knockout; Ldlr−/− or Ldlr+/−) were used.

Adeno-associated virus serotype 8 (AAV8) has been demonstrated to appropriately target genes for specific expression in liver. The murine Sort1 cDNA (Origene, MR210834) was subcloned into a specialized vector for use by the University of Pennsylvania's Penn Vector Core for production of AAV8 viral particles expressing Sort1. The virus was produced with a chimeric packaging construct in which the AAV2 rep gene was fused with the cap gene of AAV serotype 8. Empty AAV8 viral particles were also provided by the Penn Vector Core. AAV8 vector encoding the murine Sort1 gene driven by a liver-specific promoter (thyroglobulin) was delivered to mouse liver via intraperitoneal injection. A null virus was used as the control. After fasting for 4 hours, mice received with 1×10¹² viral particles of null AAV or 1×10¹² viral particles of AAV encoding Sort1 in PBS via intraperitoneal injection.

Chemically synthesized small interfering RNA (siRNA)-mediated knockdown of Apob or Pcsk9 in liver has been successful in determining the effects of these genes on plasma lipid levels. A similar approach was used to reduce Sort1 expression in mouse liver. SiRNA duplexes that effected >90% knockdown of Sort1 expression in cells. One chemically modified duplex with a low half-maximal inhibitory concentration (IC₅₀) that did not induce cytokines in a human peripheral blood mononuclear cell assay was selected for large-scale preparation in a lipidoid formulation optimized for liver-specific delivery and injection into mouse tail veins. As a negative control in some experiments, a chemically modified, non-immunostimulatory siRNA duplex specific for the firefly luciferase gene was used.

Total plasma cholesterol and alanine aminotransferase (ALT) were measured enzymatically on a Cobas Mira autoanalyzer (Roche Diagnostic Systems). Pooled plasma from each experimental group (140 μL) was separated by fast protein liquid (FPLC) gel filtration. Cholesterol plate assays were performed on FPLC fractions using the Infinity cholesterol reagent. Individual plasma samples were sent for nuclear magnetic resonance (NMR) lipoprotein measurement (LipoScience). To study hepatic VLDL secretion, mice were prebled by retro-orbital bleeding and administered an intraperitoneal injection of 400 μL of 1 mg/g Pluronic F-127 detergent resuspended in PBS. Serial retro-orbital bleeds were performed at one, two, and four hours after injection. Plasma samples were individually subjected to triglyceride measurements by analytical chemistry and pooled together by experimental condition and sent for NMR analysis for VLDL measurement (LipoScience).

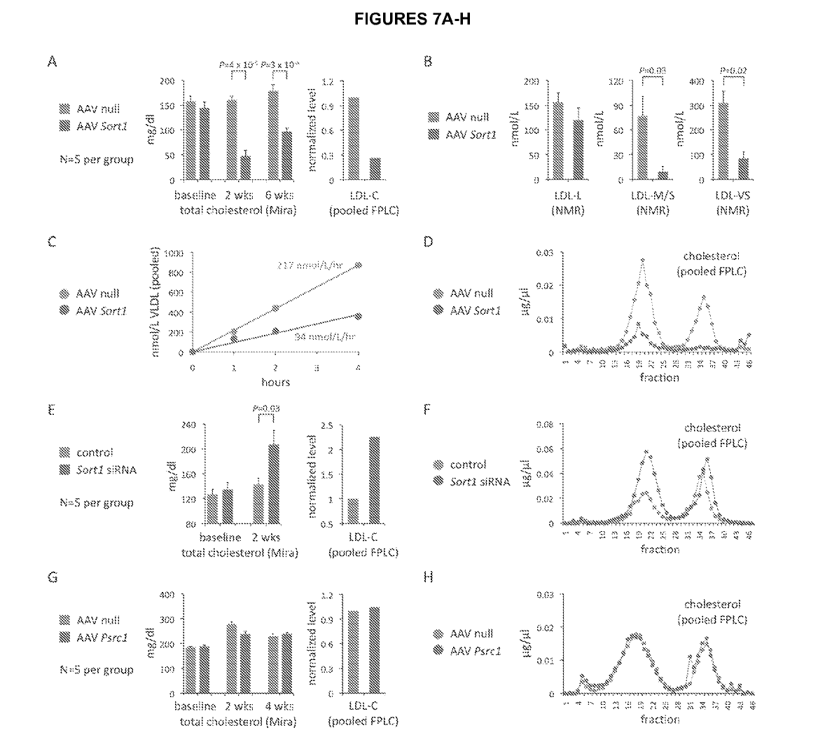

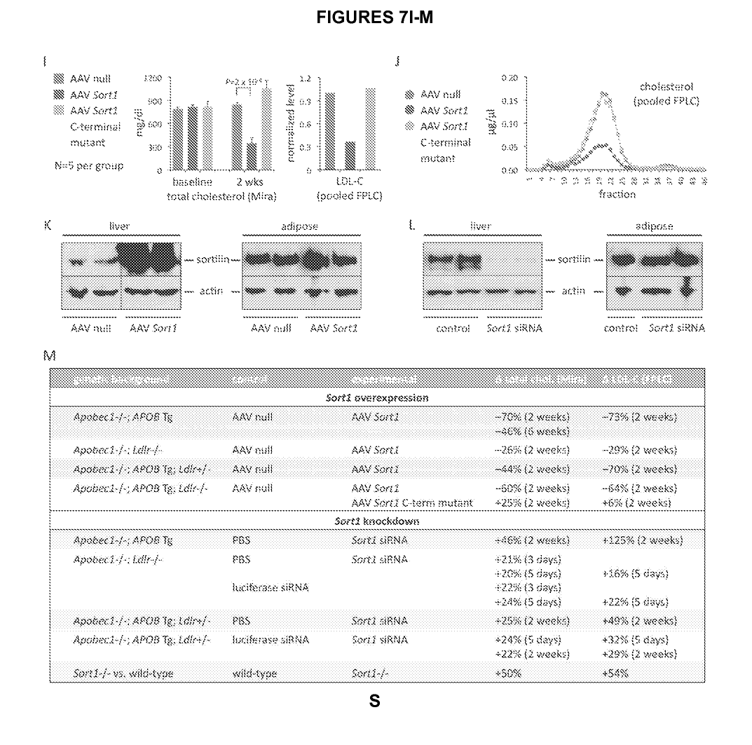

The Sort1 AAV resulted in increased sortilin levels in liver, but no change in sortilin abundance in adipose was observed (FIG. 7K). Use of these viral vectors did not result in elevation in ALT levels. Two weeks after AAV injection, plasma lipids were measured by analytical chemistry and plasma lipoproteins by FPLC, and six weeks after injection plasma lipids and lipoproteins were measured with analytical chemistry and NMR, respectively. When compared with mice injected with null virus, Sort1-overexpressing Apobec1−/−; APOB Tg mice showed a marked decrease in total plasma cholesterol (70% reduction at two weeks, 46% reduction at six weeks) and LDL-C (73% reduction at two weeks) (FIG. 7A, 7D); consistent results were seen in three other mouse backgrounds (FIG. 7I, 7J, 7M). At six weeks the mice displayed a 73% reduction in very small LDL particles and an 88% reduction in medium small LDL particles (FIG. 7B), resulting in an increase in LDL peak particle size (20.9 nm vs. 22.0 nm, P=0.05).

To study hepatic VLDL secretion, Pluronic F-127 detergent was administered to the AAV-injected mice. Triglyceride levels in individual mice were measured and NMR measurements of pooled plasma samples were taken serially at baseline, one hour, two hours, and four hours after injection of the detergent. A 57% decrease in the rate of VLDL secretion was observed (FIG. 7C), as was a similar decrease in the rate of triglyceride secretion in Sort1-overexpressing mice.

Sort1 siRNA achieved 70%-80% reduction in Sort1 expression in liver, confirmed to be due to siRNA-mediated cleavage, as well as reduced sortilin levels in liver with no change in adipose (FIG. 7L). Sort1 knockdown in Apobec1−/−; APOB Tg mice resulted in a 46% increase in total cholesterol compared to control mice at two weeks, with a more than two-fold increase in LDL-C (FIG. 7E, F). Consistent results were seen in two other mouse backgrounds (FIG. 7M).

Besides SORT1, PSRC1 displayed the greatest differential expression in human liver by 1p13 rs12740374 (see FIG. 1C). To determine whether PSRC1 also contributes to plasma lipid levels, an AAV8 vector encoding the murine Psrc1 gene was used for mouse liver overexpression. No significant changes in either total cholesterol or LDL-C levels were observed (FIG. 7G, 7H). Psrc1 mRNA could not be detected in wild-type mouse liver, preempting knockdown experiments.

In summary, hepatic Sort1 overexpression in mouse liver decreased plasma LDL-C concentration, plasma LDL particle concentrations, and secretion of VLDL. The gain-of-function studies in mice were concordant with the genetic findings in human cohorts, in whom the minor haplotype of the 1p13 locus is associated with both increased liver SORT1 expression and decreased LDL particle concentrations (FIG. 1A, 1C). Together, these studies indicate that SORT1 is the causal gene in the 1p13 locus.

### Example 5

### Sortilin Affects ApoB Processing in Liver Cells

To assay for the effects of sortilin on lipoprotein processing pathways in hepatocytes, labeling experiments using primary mouse hepatocytes were performed. Mice of the Apobec−/−; APOB Tg; Ldlr+/− or Apobec−/−; Ldlr−/− background that had been administered AAV vectors or siRNAs were used as the source of primary hepatocytes for all experiments. Mice were anesthetized with 2,2,2-tribromoethanol and then dissected to expose the liver, portal vein, and inferior vena cava. A catheter was inserted into the portal vein and sutured in place. The livers were perfused with buffer for five minutes to remove all red blood cells, followed by digestion in situ by running digestion media through the catheter for 15 minutes. The livers were transferred to 10 mm dishes with 15 mL of hepatocyte wash media and run through a mesh into 50 mL conical tubes to separate the cells. The cells were centrifuged at 50 g at 4° C. to remove Kupffer cells. The hepatocyte pellets were washed twice with hepatocyte wash media and resuspended in 25 mL PBS+25 ml of Percoll solution (45 mL Percoll+5 mL 10× PBS+100 μL of 1M HEPES). The cells were then centrifuged at 115 g for five minutes at 4° C. to pellet the viable hepatocytes. The hepatocytes were resuspended in Hepatozyme media +10% FBS +1% amino acids and plated at one million cells per well. A subset of the cells was analyzed for sortilin and actin protein expression.

For labeling experiments, cells were switched to cystine/methionine-free DMEM with 1% FBS, 1% antibiotics/antimycotics, and 0.4 mM oleic acid for one hour, followed by addition of 200 μCi/well of ³⁵S-methionine/cysteine. In some cases, 10 μM E64d was added prior to the labeling. After three hours, media from the cells were harvested, and apoB was immunoprecipitated with the antibody ab20737 (Abcam). The immunoprecipitate was subjected to SDS-PAGE, and the gel was exposed to film at −80° C. for two weeks. Relative secreted apoB levels were determined by quantitation of appropriately sized bands by densitometry. To determine relative total secreted protein levels, 50 μL of 2 mg/mL BSA and 25 μL of 50% trichloroacetic acid (TCA) were added to 50 μL of harvested media, followed by incubation on ice for 20 minutes. The samples were centrifuged for 15 minutes, and the pellets were washed with 1 mL of 50% TCA and resuspended by boiling in 1 ml of 0.2 M NaOH. 200 μL of the NaOH suspension was analyzed in a scintillation counter for ³⁵S counts.

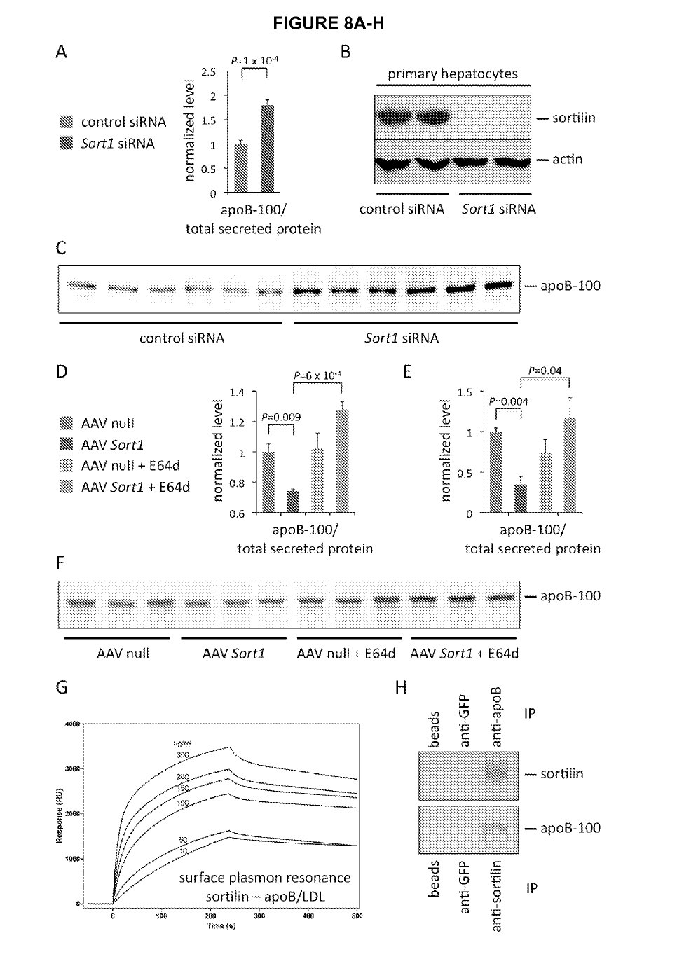

In assays in which Sort1 expression was knocked down, a significant increase in apoB secretion was observed (FIG. 8A-8C). In hepatocytes with Sort1 overexpression, there was decreased apoB secretion (FIG. 8D-8F).

Because sortilin has been reported to bind other apolipoproteins (see Jacobsen et al., J. Biol. Chem. 276: 22788-22796 (2001); Nilsson et al., J. Biol. Chem. 283: 25920-25927 (2008), assays were performed to determine whether sortilin might regulate apoB via direct binding. Surface plasmon resonance experiments demonstrated binding of recombinant sortilin to apoB in LDL particles with K_{d}˜2 nM but no binding to high-density lipoprotein (HDL) particles (which harbor no apoB) (FIG. 8G). For immunoprecipitation assays, confluent SORT1-overexpressing HuH-7 cells were lysed in 1% Triton-X, protease inhibitors, and 3 mM dithiobis(succinimidyl propionate) for 30 minutes on ice. Crosslinking was stopped with 50 mM Tris (pH 7.5). Cell nuclei were pelleted by centrifugation at 13,500 rpm for 30 minutes. The remaining cell extracts were incubated overnight at 4° C. alone or with an isotope matched GFP antibody, a human apoB antibody (Abcam), or a human sortilin antibody. Protein A agarose beads were added to the extract. Bead eluates (with lysis buffer, described above) were subjected to Western blotting and probed for apoB-100 or sortilin (Abcam ab20737, ab16640). ApoB-100 was immunoprecipitated with antibody specific to sortilin, and sortilin was immunoprecipitated with antibody specific to apoB-100 (FIG. 8H).

Production of apoB-containing lipoproteins in hepatocytes is highly regulated, with posttranslational degradation being the most important factor regulating the rate of VLDL apoB secretion. Degradation of apoB occurs at various intracellular sites, grouped as ER-associated degradation (ERAD) and post-ER, presecretory proteolysis (PERPP). The former is mediated by cytoplasmic proteasomes and is well understood; the latter occurs in the endolysosomal compartment and is less characterized. Hypothesizing that sortilin reduces secreted apoB levels by targeting apoB for endolysosomal degradation, and recognizing that sortilin harbors a C-terminal endolysosomal sorting motif, the ability of a specific inhibitor of endolysosomal cathepsin (E64d, which has also been shown to be an inhibitor of PERPP) to modulate the effects of sortilin overexpression on apoB was tested. Treatment of Sort1-overexpressing hepatocytes with E64d eliminated the reduction in apoB secretion by Sort1 observed in untreated cells in labeling experiments (FIG. 8D-8F).

An AAV8 vector encoding a truncated Sort1 gene lacking the C-terminal endolysosomal sorting motif was used for mouse liver overexpression. Despite abundant protein expression, it had no effect on plasma lipid levels, in direct contrast to wild-type Sort1 (FIG. 7I, 7J).

In sum, these observations are consistent with the hypothesis that sortilin binds apoB in VLDL and shunts the presecretory apoB-containing lipoprotein particles from the ER/Golgi compartments to lysosomes, preempting the secretion of the particles and resulting in their proteolysis.

### Example 6

### Regulatory Pathway for Lipoproteins Involving a Common Variant in the 1p13 Locus and SORT1

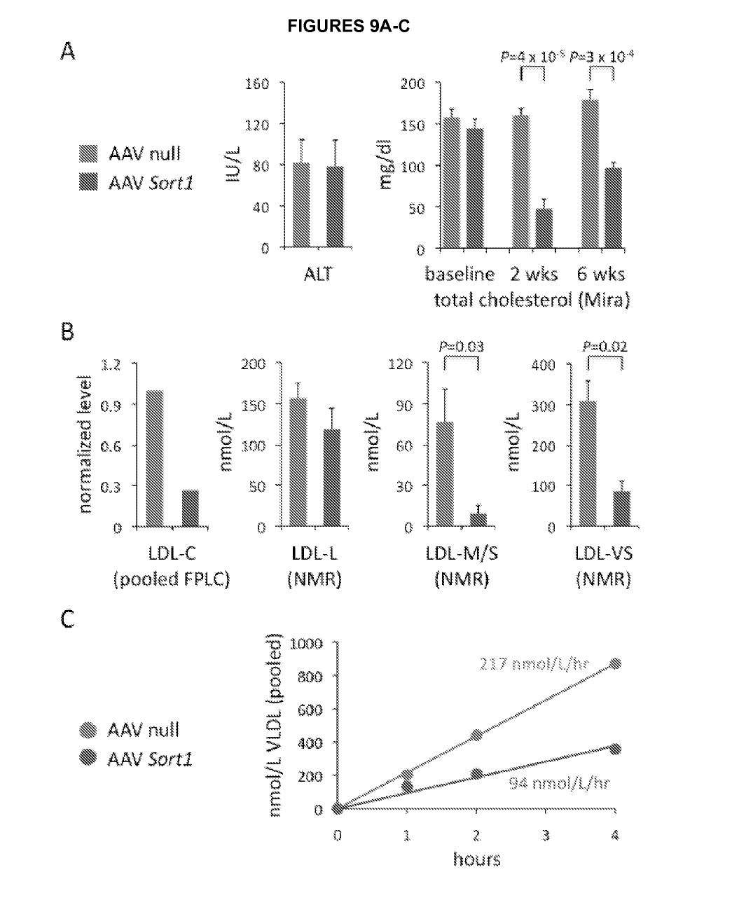

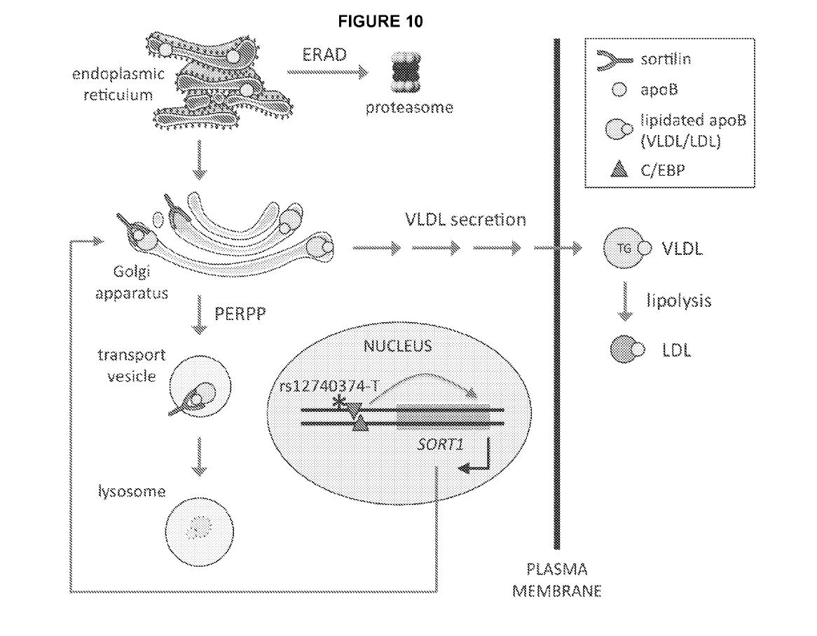

Through a series of studies in human cohorts, mice, and hepatocytes, evidence that a single noncoding DNA variant at the chromosome 1p13 locus influences LDL-C and MI risk via liver-specific transcriptional regulation of the SORT1 gene was provided. It has been shown that (1) the minor haplotype at the 1p13 locus is associated with decreased plasma LDL and VLDL particle subclass concentrations in humans, particularly very small LDL particles, (2) the minor allele of rs12740374 in the 1p13 locus creates a C/EBP consensus binding site, through which a transcription factor can enhance gene expression, (3) the minor haplotype is correlated with a four-fold increase in SORT1 expression specifically in human liver, (4) increased SORT1 expression in cultured human hepatocytes results in decreased intracellular and secreted apoB, and (5) increased Sort1 expression in mouse liver results in decreased total plasma cholesterol, decreased plasma LDL-C and LDL lipoprotein particle concentrations, particularly small LDL particles, and decreased apoB/VLDL particle secretion. These results suggest a novel biological pathway of lipoprotein regulation (FIG. 9).

The clinical importance of this pathway is defined by the 40% difference in myocardial infarction (MI) risk between homozygotes of the alternative 1p13 haplotypes. As the 1p13 minor allele frequency is about 30% in individuals of European descent and is common in other ethnicities including African Americans, Hispanics, Asian Indians, and Chinese, this locus is an important genetic determinant of population-wide risk for MI.

These experiments suggest that sortilin may promote pre-secretory apoB/VLDL degradation by diverting apoB-containing particles away from the secretory pathway and into the endolysosomal compartment as part of the PERPP pathway (FIG. 9). Among LDL particles, the largest change per 1p13 genotype was observed with the LDL-VS subclass. Little is known about the genesis of plasma LDL-VS particles in humans, although it has been proposed that LDL-VS particles are derived by rapid extrahepatic lipolysis of very large, triglyceride-rich VLDL particles rather than smaller, triglyceride-poor VLDL particles, and that PERPP acts to preferentially reduce secretion of larger VLDL particles. It is possible that sortilin serves a “quality control” function in post-ER VLDL secretion by preferentially shunting nascent large VLDL particles, which may be particularly susceptible to alterations arising from assembly, transport, or oxidation, for degradation in lysosomes and resulting in decreased secretion of these particles and, consequently, decreased substrate for LDL-VS production.

In conclusion, these results nominate the causal gene at the 1p13 locus for LDL-C and MI as SORT1 and the sortilin pathway as a promising new target for therapeutic intervention in the reduction of LDL-C and prevention of MI. They also provide insights into mechanisms by which common noncoding genetic variants can lead to clinical phenotypes, rather than simply being markers for disease.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of predicting a human patient's likelihood of developing a cardiovascular disorder, the method comprising determining the identity of at least one allele of a single nucleotide polymorphism at rs12740374 in the subject, wherein the presence of an allele associated with a cardiovascular disorder indicates that the subject has an increased risk of developing the cardiovascular disorder, and wherein the presence of an alternative allele indicates that the subject has a decreased risk of developing the cardiovascular disorder.

2. The method of claim 1, wherein the cardiovascular disorder is myocardial infarction or elevated levels of low-density lipoprotein cholesterol (LDL-C).

3. The method of claim 1, wherein the allele associated with a cardiovascular disorder is a “G” at nucleotide 27 of SEQ ID NO:11.

4. The method of claim 1, wherein determining the identity of an allele comprises obtaining a sample comprising DNA from the patient, and determining identity of a nucleotide at rs12740374.

5. The method of claim 4, wherein determining the identity of the nucleotide comprises contacting the sample with a probe specific for a selected allele of rs12740374, and detecting the formation of complexes between the probe and the selected allele of rs12740374, wherein the formation of complexes between the probe and the test marker indicates the presence of the selected allele in the sample.

6. The method of claim 1, further comprising selecting a treatment method based on the presence of an allele associated with a cardiovascular disorder at rs12740374.

7. The method of claim 6, wherein the treatment comprises administration of a medicament selected from the group consisting of a hypolipidemic medication, a vasodilating compound, an anticoagulant, and sublingual glyceryl trinitrate, or any combination thereof.

8. The method of claim 6, further comprising administering the selected treatment to the subject.

9. The method of claim 1, further comprising recording the identity of the allele in a tangible medium.

10. The method of claim 9, wherein the tangible medium comprises a computer readable disk, a solid state memory device, or an optical storage device.
